(19) United States
(12) Patent Application Publication
(10) Pub. No.: US 2024/0390287 A1
(43) Pub. Date: Nov. 28, 2024

(54) HIGH-THROUGHPUT METHODS FOR PREPARING LIPID NANOPARTICLES AND USES THEREOF

(71) Applicant: GENENTECH, INC., South San Francisco, CA (US)

(72) Inventors: Yuchen FAN, South San Francisco, CA (US); Chun-Wan YEN, South San Francisco, CA (US); Ke ZHANG, South San Francisco, CA (US)

(73) Assignee: GENENTECH, INC., South San Francisco, CA (US)

(21) Appl. No.: 18/642,305

(22) Filed: Apr. 22, 2024

Related U.S. Application Data
(63) Continuation of application No. PCT/US2022/078710, filed on Oct. 26, 2022.
(60) Provisional application No. 63/272,136, filed on Oct. 26, 2021.

(51) Int. Cl. A61K 9/51; A61K 31/7105

(52) U.S. Cl. CPC A61K 9/5192; A61K 9/5123; A61K 31/7105

(57) ABSTRACT

Provided herein are high throughput methods for optimizing and manufacturing various lipid nanoparticle (LNP) compositions and uses thereof. In some embodiments, the present disclosure provides a high-throughput screening method for manufacturing a LNP composition comprising, obtaining at least two intermixable solutions comprising a payload and a plurality of molecules capable of self-assembly, and mixing said at least two solutions under a set of controlled conditions, by which injection sequence, speed, volume, phase ratio and mixing duration are varied. In various embodiments, the present disclosure enables optimal encapsulation efficiency, particle size distribution, purification and particle recovery rate, and formulation stability to be determined. The methods disclosed herein enable efficient optimization of manufacturing conditions for preparation of LNP-based therapeutics.

### CROSS REFERENCE TO RELATED APPLICATIONS

This application is a continuation of International Application No. PCT/US2022/078710, filed Oct. 26, 2022, which claims benefit of priority of U.S. Provisional Application No. 63/272,136, filed Oct. 26, 2021, the contents of which are hereby incorporated by reference in their entirety.

### BACKGROUND

Lipid nanoparticles (LNPs) have been widely developed as biocompatible and stable pharmaceutical delivery platforms. The lipids used to prepare lipid nanoparticles are usually physiological lipids (biocompatible and biodegradable) with low toxicity. The physicochemical diversity and biocompatibility of lipids and their ability to enhance oral bioavailability of drugs have made lipid nanoparticles very attractive carriers for drug delivery. Moreover, the lipid-based formulations can positively influence drug absorption in a number of ways including: increasing solubilization capacity, preventing drug precipitation on intestinal dilution, enhancing membrane permeability, inhibiting efflux transporters, reducing CYP enzymes, enhancing chylomicron production and lymphatic transport. LNPs are the leading non-viral carriers for the siRNA delivery and are employed in 70% of nanomedicine clinical trials as of 2019. Anselmo S et al., 2019, Bioeng. Transl. Med. 4(3):e10143.

Lipid-based nanocarriers bring additional challenges in quality control of drug products partially due to their complicated physicochemical properties. According to the guidance on liposome drug products recently published by the U.S. FDA, these formulations should be specified for quality attributes including the particle structure and size distribution, physicochemical properties of the particle surface, lipid content, amount of the free API and encapsulation efficiency, and physical and chemical stability. Different preparation conditions and parameters may impact the quality attributes of LNP formulations. For example, lipid compositions, in particular incorporation of different amounts and/or molecular weights of the PEGylated lipid, significantly impacted colloidal stability, cellular uptake, and pharmacokinetics of liposomes (see, e.g. Allen et al., 1991, Biochem Biophys Acta, 1066(1):29-36; Garbuzenko et al., 2005, Chem Phys Lipids, 135(2): 117-29; Immordino et al., Int J Nanomedicine 1(3) (2006) 297-315) whereas siRNA or ASO loading could be controlled by charge-mediated interactions with cationic lipids. Schroeder et al., 2010, J Intern Med 267(1): 9-21; Cullis et al., 2017, Mol Ther 25(7):1467-1475. The downstream performance of LNPs is also highly governed by their quality attributes. Screening of various levels of these parameters therefore highly demands a high-throughput approach with facile procedures and multiple analytical outputs.

LNP structure and cargo delivery are regulated by four major components: ionizable lipids, helper phospholipids, cholesterol, and polyethylene glycol-lipids (PEG-lipids). Cationic ionizable lipids promote the encapsulation of negatively charged nucleic acids during LNP formulation and aid in cytosolic delivery of cargo at endosomal pH range 5.5-6.5. Helper lipids and cholesterol increase structural stability, promote membrane fusion, and enhance endosomal escape of the LNPs. The effect of PEG-lipid addition is multi-faceted and contributes to what has been termed the ‘PEG-dilemma’. PEG-lipids are necessary to control particle size during self-assembly and prevent particle aggregation. However, the hydrophilic PEG corona can hinder interactions between the particle surface and the lipophilic cell membrane, resulting in poor cellular internalization. The presence of PEG can also prevent surface binding of transport proteins necessary for LNP cellular internalization via receptor-mediated endocytosis. Furthermore, PEG-lipids also prolong the in vivo LNP circulation time by acting as a steric barrier to the adsorption of plasma proteins, including opsonins. While extending half-life increases therapeutic exposure, increasing LNP circulation can induce anti-PEG antibody generation leading to adverse allergic reactions.

Thus, achieving effective intracellular delivery is highly dependent on understanding the effect of multivariate PEG-lipids to overcome the PEG dilemma. Previous studies indicate tuning PEG size, architecture, content, carbon tail type, and length modulates LNP efficacy.

### SUMMARY OF THE INVENTION

To address the needs of screening and optimization of lipid-based nanomedicines, the present disclosure provides for a high-throughput screening (HTS) workflow for the preparation of such lipid-based nanoparticles encapsulating various therapeutic payloads. In various embodiments, the present invention provides an optimized solvent-injection method for facile self-assembly of LNPs using a robotic liquid handler. In various embodiments, optimal lipid composition, total lipid concentration, and loading amount of a payload are described.

In various embodiments, the present disclosure relates to an optimized high-throughput screening method for manufacturing a lipid nanoparticle (LNP) preparation comprising: a. obtaining a first solution comprising an aqueous phase; b. obtaining a second solution comprising an organic phase and a plurality of molecules capable of self-assembly, and wherein said first and second solutions are intermixable; c. dissolving at least one payload molecule into either the first or second solution; d. using a robotic liquid handler to prepare and dispense said phases with varied compositions into a plurality of wells; e. mixing said first and second solutions to obtain lipid nanoparticles encapsulating said payload using said robotic liquid handler under conditions suitable for LNP formation; wherein at least one of the following conditions are varied amongst different wells: type of self-assembly molecule, composition ratio of said self-assembly molecule; ratio and/or concentration of said self-assembly molecule to said payload, the selection of phase, buffer type and pH, the injection sequence, injection speed, mixing speed, volume, phase ratio, injection duration, and mixing duration; f. measuring at least one of the following: encapsulation efficiency, particle size distribution, purification and particle recovery rate, and formulation stability of said LNPs; g. determining the optimal parameters for manufacturing said LNP preparation; and h. manufacturing said LNP preparation based on said optimal parameters.

As noted herein, Lipid Nanoparticles (LNPs) are increasingly utilized to improve the delivery efficiency and therapeutic efficacy of nucleic acids. It has been found that various formulation parameters can affect the quality attributes of these nanoparticle formulations. There is, however, currently a lack of effective systemic screening approaches to address these challenges. In accordance with the invention, an automated high-throughput screening (HTS) workflow has been developed for streamlined preparation and analytical characterization of LNPs loaded with, e.g., antisense oligonucleotides (ASOs). In accordance with the invention, this characterization can be performed in a full 96-well plate within as little as 3 hours.

In accordance with the invention, ASO-loaded LNPs were formulated by an automated solvent-injection method using a robotic liquid handler, and assessed for particle size distribution, encapsulation efficiency, and stability with different formulation compositions and ASO loadings. The results described herein indicate that the PEGylated lipid content significantly affected the particle size distribution, while the ionizable lipid/ASO charge ratio impacted the encapsulation efficiency of the ASOs. Furthermore, results from our HTS approach correlated with those from the state-of-the-art scale-up method using a microfluidic formulator, thus providing a novel method for robust formulation development and design of experiment methods. This method can reduce material usage by ˜10-fold, thereby improving analytical outputs and accumulation of information by ˜100 fold.

A novel HTS workflow has been developed to prepare ASO-LNPs and analyze the relationship between their PEG-lipid content and particle size distribution. Using a high-throughput liquid handler, libraries of varying ASO-LNPs containing multiple PEG-lipids across the phosphoglyceride, diglyceride, and ceramide families were made and formulated at varying PEG-lipid molar ratios (1-5 mol %). The impact of PEG-lipid parameters including molecular weight (mw), carbon-tail length, and molar ratios is described herein. Additionally, a number of additional PEG-lipid variables are described herein, including PEG architecture, lipid tail saturation, PEG-lipid charge, and linker chemistry.

In various embodiments, the payload is an oligonucleotide. In various embodiments, the oligonucleotide is an antisense molecule. In various embodiments, the oligonucleotide is a siRNA. In various embodiments, the oligonucleotide is a shRNA. In various embodiments, the oligonucleotide is between about 10 to about 30 nucleotides in length. In various embodiments, the payload is an mRNA. In various embodiments, the size of mRNA is about 500-3000 nucleotides. In various embodiments, the payload is a polypeptide. In various embodiments, said polypeptide is between about 1,000 Da and about 10,000 Da. In various embodiments, the payload is a small molecule. In various embodiments, the small molecule is between about 100 Da and 1000 Da.

In various embodiments, the payload is dissolved in the first solution. In various embodiments, the payload is dissolved in the second solution. In various embodiments, the first solution is an aqueous buffer. In various embodiments, the first solution comprises pH- and osmolality-controlled buffers. In various embodiments, the organic phase of the second solution comprises methanol. In various embodiments, the organic phase of the second solution comprises ethanol.

In various embodiments, the self-assembling molecules include at least a lipid component comprised of at least one species of lipid molecule. In various embodiments, the at least one species of lipid molecule is selected from a cationic or ionizable lipid species, a non-cationic lipid species, and a phospholipid species. In various embodiments, said second solution comprises more than one type of lipid. In various embodiments, the total concentration of lipid is varied. In various embodiments, the total concentration of lipid is varied between about 0.4 and about 4 mM. In various embodiments, the percentage of lipids that are PEGylated is varied. In various embodiments, the percentage of lipids that are PEGylated are varied between about 0.5% to about 5% of the total lipid composition. In various embodiments, the payload's N:P ratio is varied. In various embodiments, the N:P ratio is varied between about 0.5 to about 5.

In various embodiments, the LNP is a polymer lipid nanoparticle. In various embodiments, the LNP is a liposome. In various embodiments, the LNP is a lipoprotein nanoparticle. In various embodiments, said first solution is injected into said second solution. In various embodiments, said second solution is injected into said first solution. In various embodiments, the optimal parameters are those which produce an encapsulation efficiency of the payload greater than 80%. In various embodiments, the optimal parameters are those which produce a LNP with a mean diameter of 80-200 nm, having a unimodal size distribution, and a polydispersity of less than about 30%. In various embodiments, the LNPs maintain a similar size distribution and payload encapsulation for at least one month under storage in solution at 4 degrees Celsius.

In various embodiments, the present disclosure relates to a high-throughput method for optimizing the process for manufacturing a lipid nanoparticle (LNP) preparation comprising: a. obtaining a first solution comprising an aqueous phase; b. obtaining a second solution comprising an organic phase and a plurality of molecules capable of self-assembly, and wherein said first and second solutions are intermixable; c. dissolving at least one payload molecule into either the first or second solution; d. using a robotic liquid handler to prepare and dispense said phases with varied compositions into a plurality of wells; e. mixing said first and second solutions to obtain lipid nanoparticles encapsulating said payload using said robotic liquid handler under conditions suitable for LNP formation; wherein at least one of the following conditions are varied amongst different wells: type of self-assembly molecule, composition ratio of said self-assembly molecule; ratio and/or concentration of said self-assembly molecule to said payload, the selection of phase, buffer type and pH, the injection sequence, injection speed, mixing speed, volume, phase ratio, injection duration, and mixing duration; f. measuring at least one of the following: encapsulation efficiency, particle size distribution, purification and particle recovery rate, and formulation stability of said LNPs; g. determining the optimal parameters for manufacturing said LNP preparation; and h. manufacturing said LNP preparation based on said optimal parameters.

In various embodiments, the payload is an oligonucleotide. In various embodiments, the oligonucleotide is an antisense molecule. In various embodiments, the oligonucleotide is a siRNA. In various embodiments, the oligonucleotide is a shRNA. In various embodiments, the oligonucleotide is between about 10 to about 30 nucleotides in length. In various embodiments, the payload is an mRNA. In various embodiments, the size of mRNA is about 1 kb to about 2 kb. In various embodiments, the payload is a polypeptide. In various embodiments, said polypeptide is between about 1,000 Da and about 10,000 Da. In various embodiments, the payload is a small molecule. In various embodiments, the small molecule is between about 100 Da and 1000 Da.

In various embodiments, the payload is dissolved in the first solution. In various embodiments, the payload is dissolved in the second solution. In various embodiments, the first solution is an aqueous buffer. In various embodiments, the first solution comprises pH- and osmolality-controlled buffers. In various embodiments, the organic phase of the second solution comprises methanol. In various embodiments, the organic phase of the second solution comprises ethanol.

In various embodiments, the self-assembling molecules include at least a lipid component comprised of at least one species of lipid molecule. In various embodiments, the at least one species of lipid molecule is selected from a cationic lipid species, a non-cationic lipid species, and a phospholipid species. In various embodiments, said second solution comprises more than one type of lipid. In various embodiments, the total concentration of lipid is varied. In various embodiments, the total concentration of lipid is varied between about 0.4 and about 4 mM. In various embodiments, the percentage of lipids that are PEGylated is varied. In various embodiments, the percentage of lipids that are PEGylated are varied between about 0.5% to about 5% of the total lipid composition. In various embodiments, the payload's N:P ratio is varied. In various embodiments, the N:P ratio is varied between about 0.5 to about 5.

In various embodiments, the LNP is a polymer lipid nanoparticle. In various embodiments, the LNP is a liposome. In various embodiments, the LNP is a lipoprotein nanoparticle. In various embodiments, said first solution is injected into said second solution. In various embodiments, said second solution is injected into said first solution. In various embodiments, the optimal parameters are those which produce an encapsulation efficiency of the payload greater than 80%. In various embodiments, the optimal parameters are those which produce a LNP with a mean diameter of 80-200 nm, having a unimodal size distribution, and a polydispersity of less than about 30%. In various embodiments, the LNPs maintain a similar size distribution and payload encapsulation for at least one month under storage in solution at 4 degrees Celsius.

In various embodiments, the present disclosure relates to an optimized high-throughput method for encapsulating a payload in a liquid nanoparticle (LNP) preparation comprising: a. obtaining a first solution comprising an aqueous phase; b. obtaining a second solution comprising an organic phase and a plurality of molecules capable of self-assembly, and wherein said first and second solutions are intermixable; c. dissolving at least one payload molecule into either the first or second solution; d. using a robotic liquid handler to prepare and dispense said phases with varied compositions into a plurality of wells; e. mixing said first and second solutions to obtain lipid nanoparticles encapsulating said payload using said robotic liquid handler under conditions suitable for LNP formation; wherein at least one of the following conditions are varied amongst different wells: type of self-assembly molecule, composition ratio of said self-assembly molecule; ratio and/or concentration of said self-assembly molecule to said payload, the selection of phase, buffer type and pH, the injection sequence, injection speed, mixing speed, volume, phase ratio, injection duration, and mixing duration; f. measuring at least one of the following: encapsulation efficiency, particle size distribution, purification and particle recovery rate, and formulation stability of said LNPs; g. determining the optimal parameters for manufacturing said LNP preparation; and h. manufacturing said LNP preparation based on said optimal parameters.

In various embodiments, the payload is an oligonucleotide. In various embodiments, the oligonucleotide is an antisense molecule. In various embodiments, the oligonucleotide is a siRNA. In various embodiments, the oligonucleotide is a shRNA. In various embodiments, the oligonucleotide is between about 10 to about 30 nucleotides in length. In various embodiments, the payload is an mRNA. In various embodiments, the size of mRNA is about 1 kb to about 2 kb. In various embodiments, the payload is a polypeptide. In various embodiments, said polypeptide is between about 1,000 Da and about 10,000 Da. In various embodiments, the payload is a small molecule. In various embodiments, the small molecule is between about 100 Da and 1000 Da.

In various embodiments, the payload is dissolved in the first solution. In various embodiments, the payload is dissolved in the second solution. In various embodiments, the first solution is an aqueous buffer. In various embodiments, the first solution comprises pH- and osmolality-controlled buffers. In various embodiments, the organic phase of the second solution comprises methanol. In various embodiments, the organic phase of the second solution comprises ethanol.

In various embodiments, the self-assembling molecules include at least a lipid component comprised of at least one species of lipid molecule. In various embodiments, the at least one species of lipid molecule is selected from a cationic lipid species, a non-cationic lipid species, and a phospholipid species. In various embodiments, said second solution comprises more than one type of lipid. In various embodiments, the total concentration of lipid is varied. In various embodiments, the total concentration of lipid is varied between about 0.4 and about 4 mM. In various embodiments, the percentage of lipids that are PEGylated is varied. In various embodiments, the percentage of lipids that are PEGylated are varied between about 0.5% to about 5% of the total lipid composition. In various embodiments, the payload's N:P ratio is varied. In various embodiments, the N:P ratio is varied between about 0.5 to about 5.

In various embodiments, the LNP is a polymer lipid nanoparticle. In various embodiments, the LNP is a liposome. In various embodiments, the LNP is a lipoprotein nanoparticle. In various embodiments, said first solution is injected into said second solution. In various embodiments, said second solution is injected into said first solution. In various embodiments, the optimal parameters are those which produce an encapsulation efficiency of the payload greater than 80%. In various embodiments, the optimal parameters are those which produce a LNP with a mean diameter of 80-200 nm, having a unimodal size distribution, and a polydispersity of less than about 30%. In various embodiments, the LNPs maintain a similar size distribution and payload encapsulation for at least one month under storage in solution at 4 degrees Celsius.

In various embodiments, the present disclosure relates to a method of administering a LNP preparation to a patient in need thereof, wherein said LNP preparation is manufactured by: a. obtaining a first solution comprising an aqueous phase; b. obtaining a second solution comprising an organic phase and a plurality of molecules capable of self-assembly, and wherein said first and second solutions are intermixable; c. dissolving at least one payload molecule into either the first or second solution; d. using a robotic liquid handler to prepare and dispense said phases with varied compositions into a plurality of wells; e. mixing said first and second solutions to obtain lipid nanoparticles encapsulating said payload using said robotic liquid handler under conditions suitable for LNP formation; wherein at least one of the following conditions are varied amongst different wells: type of self-assembly molecule, composition ratio of said self-assembly molecule; ratio and/or concentration of said self-assembly molecule to said payload, the selection of phase, buffer type and pH, the injection sequence, injection speed, mixing speed, volume, phase ratio, injection duration, and mixing duration; f. measuring at least one of the following: encapsulation efficiency, particle size distribution, purification and particle recovery rate, and formulation stability of said LNPs; g. determining the optimal parameters for manufacturing said LNP preparation; and h. manufacturing said LNP preparation based on said optimal parameters.

In various embodiments, the payload is an oligonucleotide. In various embodiments, the oligonucleotide is an antisense molecule. In various embodiments, the oligonucleotide is a siRNA. In various embodiments, the oligonucleotide is a shRNA. In various embodiments, the oligonucleotide is between about 10 to about 30 nucleotides in length. In various embodiments, the payload is an mRNA. In various embodiments, the size of mRNA is about 1 kb to about 2 kb. In various embodiments, the payload is a polypeptide. In various embodiments, said polypeptide is between about 1,000 Da and about 10,000 Da. In various embodiments, the payload is a small molecule. In various embodiments, the small molecule is between about 100 Da and 1000 Da.

In various embodiments, the payload is dissolved in the first solution. In various embodiments, the payload is dissolved in the second solution. In various embodiments, the first solution is an aqueous buffer. In various embodiments, the first solution comprises pH- and osmolality-controlled buffers. In various embodiments, the organic phase of the second solution comprises methanol. In various embodiments, the organic phase of the second solution comprises ethanol.

In various embodiments, the self-assembling molecules include at least a lipid component comprised of at least one species of lipid molecule. In various embodiments, the at least one species of lipid molecule is selected from a cationic lipid species, a non-cationic lipid species, and a phospholipid species. In various embodiments, said second solution comprises more than one type of lipid. In various embodiments, the total concentration of lipid is varied. In various embodiments, the total concentration of lipid is varied between about 0.4 and about 4 mM. In various embodiments, the percentage of lipids that are PEGylated is varied. In various embodiments, the percentage of lipids that are PEGylated are varied between about 0.5% to about 5% of the total lipid composition. In various embodiments, the payload's N:P ratio is varied. In various embodiments, the N:P ratio is varied between about 0.5 to about 5.

In various embodiments, the LNP is a polymer lipid nanoparticle. In various embodiments, the LNP is a liposome. In various embodiments, the LNP is a lipoprotein nanoparticle. In various embodiments, said first solution is injected into said second solution. In various embodiments, said second solution is injected into said first solution. In various embodiments, the optimal parameters are those which produce an encapsulation efficiency of the payload greater than 80%. In various embodiments, the optimal parameters are those which produce a LNP with a mean diameter of 80-200 nm, having a unimodal size distribution, and a polydispersity of less than about 30%. In various embodiments, the LNPs maintain a similar size distribution and payload encapsulation for at least one month under storage in solution at 4 degrees Celsius.

In various embodiments, the present disclosure relates to an optimized high-throughput method for encapsulating a payload in a liquid nanoparticle (LNP) preparation comprising: a. obtaining a first solution comprising an aqueous phase; b. obtaining a second solution comprising an organic phase and a plurality of molecules capable of self-assembly, and wherein said first and second solutions are intermixable; c. dissolving at least one payload molecule into either the first or second solution; d. using a robotic liquid handler to prepare and dispense said phases with varied compositions into a plurality of wells; e. mixing said first and second solutions to obtain lipid nanoparticles encapsulating said payload using said robotic liquid handler under conditions suitable for LNP formation; wherein at least one of the following conditions are varied amongst different wells: type of self-assembly molecule, composition ratio of said self-assembly molecule; ratio and/or concentration of said self-assembly molecule to said payload, the selection of phase, buffer type and pH, the injection sequence, injection speed, mixing speed, volume, phase ratio, injection duration, and mixing duration; f. measuring at least one of the following: encapsulation efficiency, particle size distribution, purification and particle recovery rate, and formulation stability of said LNPs; g. determining the optimal parameters for manufacturing said LNP preparation; and h. manufacturing said LNP preparation based on said optimal parameters.

In various embodiments, the payload is an oligonucleotide. In various embodiments, the oligonucleotide is an antisense molecule. In various embodiments, the oligonucleotide is a siRNA. In various embodiments, the oligonucleotide is a shRNA. In various embodiments, the oligonucleotide is between about 10 to about 30 nucleotides in length. In various embodiments, the payload is an mRNA. In various embodiments, the size of mRNA is about 1 kb to about 2 kb. In various embodiments, the payload is a polypeptide. In various embodiments, said polypeptide is between about 1,000 Da and about 10,000 Da. In various embodiments, the payload is a small molecule. In various embodiments, the small molecule is between about 100 Da and 1000 Da.

In various embodiments, the payload is dissolved in the first solution. In various embodiments, the payload is dissolved in the second solution. In various embodiments, the first solution is an aqueous buffer. In various embodiments, the first solution comprises pH- and osmolality-controlled buffers. In various embodiments, the organic phase of the second solution comprises methanol. In various embodiments, the organic phase of the second solution comprises ethanol.

In various embodiments, the self-assembling molecules include at least a lipid component comprised of at least one species of lipid molecule. In various embodiments, the at least one species of lipid molecule is selected from a cationic lipid species, a non-cationic lipid species, and a phospholipid species. In various embodiments, said second solution comprises more than one type of lipid. In various embodiments, the total concentration of lipid is varied. In various embodiments, the total concentration of lipid is varied between about 0.4 and about 4 mM. In various embodiments, the percentage of lipids that are PEGylated is varied. In various embodiments, the percentage of lipids that are PEGylated are varied between about 0.5% to about 5% of the total lipid composition. In various embodiments, the payload's N:P ratio is varied. In various embodiments, the N:P ratio is varied between about 0.5 to about 5.

In various embodiments, the LNP is a polymer lipid nanoparticle. In various embodiments, the LNP is a liposome. In various embodiments, the LNP is a lipoprotein nanoparticle. In various embodiments, said first solution is injected into said second solution. In various embodiments, said second solution is injected into said first solution. In various embodiments, the optimal parameters are those which produce an encapsulation efficiency of the payload greater than 80%. In various embodiments, the optimal parameters are those which produce a LNP with a mean diameter of 80-200 nm, having a unimodal size distribution, and a polydispersity of less than about 30%. In various embodiments, the LNPs maintain a similar size distribution and payload encapsulation for at least one month under storage in solution at 4 degrees Celsius.

In various embodiments, the present disclosure relates to an optimized high-throughput screening method for manufacturing a lipid nanoparticle (LNP) preparation comprising: a. obtaining a first solution comprising an aqueous phase; b. obtaining a second solution comprising an organic phase and a plurality of molecules capable of self-assembly, and wherein said first and second solutions are intermixable; c. dissolving at least one payload molecule into either the first or second solution; d. using a robotic liquid handler to prepare and dispense said phases with varied compositions into a plurality of wells; e. mixing said first and second solutions to obtain lipid nanoparticles encapsulating said payload using said robotic liquid handler under conditions suitable for LNP formation; wherein at least one of the following conditions are varied amongst different wells: type of self-assembly molecule, composition ratio of said self-assembly molecule; ratio and/or concentration of said self-assembly molecule to said payload, the selection of phase, buffer type and pH, the injection sequence, injection speed, mixing speed, volume, phase ratio, injection duration, and mixing duration; f. measuring at least one of the following: encapsulation efficiency, particle size distribution, purification and particle recovery rate, and formulation stability of said LNPs; g. determining the optimal parameters for manufacturing said LNP preparation; and h. manufacturing said LNP preparation based on said optimal parameters.

In various embodiments, the payload is an oligonucleotide. In various embodiments, the oligonucleotide is an antisense molecule. In various embodiments, the oligonucleotide is a siRNA. In various embodiments, the oligonucleotide is a shRNA. In various embodiments, the oligonucleotide is between about 10 to about 30 nucleotides in length. In various embodiments, the payload is an mRNA. In various embodiments, the size of mRNA is about 1 kb to about 2 kb. In various embodiments, the payload is a polypeptide. In various embodiments, said polypeptide is between about 1,000 Da and about 10,000 Da. In various embodiments, the payload is a small molecule. In various embodiments, the small molecule is between about 100 Da and 1000 Da.

In various embodiments, the payload is dissolved in the first solution. In various embodiments, the payload is dissolved in the second solution. In various embodiments, the first solution is an aqueous buffer. In various embodiments, the first solution comprises pH- and osmolality-controlled buffers. In various embodiments, the organic phase of the second solution comprises methanol. In various embodiments, the organic phase of the second solution comprises ethanol.

In various embodiments, the self-assembling molecules include at least a lipid component comprised of at least one species of lipid molecule. In various embodiments, the at least one species of lipid molecule is selected from a cationic lipid species, a non-cationic lipid species, and a phospholipid species. In various embodiments, said second solution comprises more than one type of lipid. In various embodiments, the total concentration of lipid is varied. In various embodiments, the total concentration of lipid is varied between about 0.4 and about 4 mM. In various embodiments, the percentage of lipids that are PEGylated is varied. In various embodiments, the percentage of lipids that are PEGylated are varied between about 0.5% to about 5% of the total lipid composition. In various embodiments, the payload's N:P ratio is varied. In various embodiments, the N:P ratio is varied between about 0.5 to about 5.

In various embodiments, the LNP is a polymer lipid nanoparticle. In various embodiments, the LNP is a liposome. In various embodiments, the LNP is a lipoprotein nanoparticle. In various embodiments, said first solution is injected into said second solution. In various embodiments, said second solution is injected into said first solution. In various embodiments, the optimal parameters are those which produce an encapsulation efficiency of the payload greater than 80%. In various embodiments, the optimal parameters are those which produce a LNP with a mean diameter of 80-200 nm, having a unimodal size distribution, and a polydispersity of less than about 30%. In various embodiments, the LNPs maintain a similar size distribution and payload encapsulation for at least one month under storage in solution at 4 degrees Celsius.

In various embodiments, the present disclosure relates to an optimized high-throughput method for encapsulating a payload in a liquid nanoparticle (LNP) preparation comprising: a. obtaining a first solution comprising an aqueous phase; b. obtaining a second solution comprising an organic phase and a plurality of molecules capable of self-assembly, and wherein said first and second solutions are intermixable; c. dissolving at least one payload molecule into either the first or second solution; d. using a robotic liquid handler to prepare and dispense said phases with varied compositions into a plurality of wells; e. mixing said first and second solutions to obtain lipid nanoparticles encapsulating said payload using said robotic liquid handler under conditions suitable for LNP formation; wherein at least one of the following conditions are varied amongst different wells: type of self-assembly molecule, composition ratio of said self-assembly molecule; ratio and/or concentration of said self-assembly molecule to said payload, the selection of phase, buffer type and pH, the injection sequence, injection speed, mixing speed, volume, phase ratio, injection duration, and mixing duration; f. measuring at least one of the following: encapsulation efficiency, particle size distribution, purification and particle recovery rate, and formulation stability of said LNPs; g. determining the optimal parameters for manufacturing said LNP preparation; and h. manufacturing said LNP preparation based on said optimal parameters.

In various embodiments, the payload is an oligonucleotide. In various embodiments, the oligonucleotide is an antisense molecule. In various embodiments, the oligonucleotide is a siRNA. In various embodiments, the oligonucleotide is a shRNA. In various embodiments, the oligonucleotide is between about 10 to about 30 nucleotides in length. In various embodiments, the payload is an mRNA. In various embodiments, the size of mRNA is about 1 kb to about 2 kb. In various embodiments, the payload is a polypeptide. In various embodiments, said polypeptide is between about 1,000 Da and about 10,000 Da. In various embodiments, the payload is a small molecule. In various embodiments, the small molecule is between about 100 Da and 1000 Da.

In various embodiments, the payload is dissolved in the first solution. In various embodiments, the payload is dissolved in the second solution. In various embodiments, the first solution is an aqueous buffer. In various embodiments, the first solution comprises pH- and osmolality-controlled buffers. In various embodiments, the organic phase of the second solution comprises methanol. In various embodiments, the organic phase of the second solution comprises ethanol.

In various embodiments, the self-assembling molecules include at least a lipid component comprised of at least one species of lipid molecule. In various embodiments, the at least one species of lipid molecule is selected from a cationic lipid species, a non-cationic lipid species, and a phospholipid species. In various embodiments, said second solution comprises more than one type of lipid. In various embodiments, the total concentration of lipid is varied. In various embodiments, the total concentration of lipid is varied between about 0.4 and about 4 mM. In various embodiments, the percentage of lipids that are PEGylated is varied. In various embodiments, the percentage of lipids that are PEGylated are varied between about 0.5% to about 5% of the total lipid composition. In various embodiments, the payload's N:P ratio is varied. In various embodiments, the N:P ratio is varied between about 0.5 to about 5.

In various embodiments, the LNP is a polymer lipid nanoparticle. In various embodiments, the LNP is a liposome. In various embodiments, the LNP is a lipoprotein nanoparticle. In various embodiments, said first solution is injected into said second solution. In various embodiments, said second solution is injected into said first solution. In various embodiments, the optimal parameters are those which produce an encapsulation efficiency of the payload greater than 80%. In various embodiments, the optimal parameters are those which produce a LNP with a mean diameter of 80-200 nm, having a unimodal size distribution, and a polydispersity of less than about 30%. In various embodiments, the LNPs maintain a similar size distribution and payload encapsulation for at least one month under storage in solution at 4 degrees Celsius.

In various embodiments, the present disclosure relates to an optimized lipid nanoparticle (LNP) manufactured by a process comprising the following steps: a. obtaining a first solution comprising an aqueous phase; b. obtaining a second solution comprising an organic phase and a plurality of molecules capable of self-assembly, and wherein said first and second solutions are intermixable; c. dissolving at least one payload molecule into either the first or second solution; d. using a robotic liquid handler to prepare and dispense said phases with varied compositions into a plurality of wells; e. mixing said first and second solutions to obtain lipid nanoparticles encapsulating said payload using said robotic liquid handler under conditions suitable for LNP formation; wherein at least one of the following conditions are varied amongst different wells: type of self-assembly molecule, composition ratio of said self-assembly molecule; ratio and/or concentration of said self-assembly molecule to said payload, the selection of phase, buffer type and pH, the injection sequence, injection speed, mixing speed, volume, phase ratio, injection duration, and mixing duration; f. measuring at least one of the following: encapsulation efficiency, particle size distribution, purification and particle recovery rate, and formulation stability of said LNPs; g. determining the optimal parameters for manufacturing said LNP preparation; and h. manufacturing said LNP preparation based on said optimal parameters.

In various embodiments, the payload is an oligonucleotide. In various embodiments, the oligonucleotide is an antisense molecule. In various embodiments, the oligonucleotide is a siRNA. In various embodiments, the oligonucleotide is a shRNA. In various embodiments, the oligonucleotide is between about 10 to about 30 nucleotides in length. In various embodiments, the payload is an mRNA. In various embodiments, the size of mRNA is about 1 kb to about 2 kb. In various embodiments, the payload is a polypeptide. In various embodiments, said polypeptide is between about 1,000 Da and about 10,000 Da. In various embodiments, the payload is a small molecule. In various embodiments, the small molecule is between about 100 Da and 1000 Da.

In various embodiments, the payload is dissolved in the first solution. In various embodiments, the payload is dissolved in the second solution. In various embodiments, the first solution is an aqueous buffer. In various embodiments, the first solution comprises pH- and osmolality-controlled buffers. In various embodiments, the organic phase of the second solution comprises methanol. In various embodiments, the organic phase of the second solution comprises ethanol.

In various embodiments, the self-assembling molecules include at least a lipid component comprised of at least one species of lipid molecule. In various embodiments, the at least one species of lipid molecule is selected from a cationic lipid species, a non-cationic lipid species, and a phospholipid species. In various embodiments, said second solution comprises more than one type of lipid. In various embodiments, the total concentration of lipid is varied. In various embodiments, the total concentration of lipid is varied between about 0.4 and about 4 mM. In various embodiments, the percentage of lipids that are PEGylated is varied. In various embodiments, the percentage of lipids that are PEGylated are varied between about 0.5% to about 5% of the total lipid composition. In various embodiments, the payload's N:P ratio is varied. In various embodiments, the N:P ratio is varied between about 0.5 to about 5.

In various embodiments, the LNP is a polymer lipid nanoparticle. In various embodiments, the LNP is a liposome. In various embodiments, the LNP is a lipoprotein nanoparticle. In various embodiments, said first solution is injected into said second solution. In various embodiments, said second solution is injected into said first solution. In various embodiments, the optimal parameters are those which produce an encapsulation efficiency of the payload greater than 80%. In various embodiments, the optimal parameters are those which produce a LNP with a mean diameter of 80-200 nm, having a unimodal size distribution, and a polydispersity of less than about 30%. In various embodiments, the LNPs maintain a similar size distribution and payload encapsulation for at least one month under storage in solution at 4 degrees Celsius.

In various embodiments, the present disclosure relates to an optimized high-throughput method for encapsulating a payload in a liquid nanoparticle (LNP) preparation comprising: a. obtaining a first solution comprising an aqueous phase; b. obtaining a second solution comprising an organic phase and a plurality of molecules capable of self-assembly, and wherein said first and second solutions are intermixable; c. dissolving at least one payload molecule into either the first or second solution; d. using a robotic liquid handler to prepare and dispense said phases with varied compositions into a plurality of wells; e. mixing said first and second solutions to obtain lipid nanoparticles encapsulating said payload using said robotic liquid handler under conditions suitable for LNP formation; wherein at least one of the following conditions are varied amongst different wells: type of self-assembly molecule, composition ratio of said self-assembly molecule; ratio and/or concentration of said self-assembly molecule to said payload, the selection of phase, buffer type and pH, the injection sequence, injection speed, mixing speed, volume, phase ratio, injection duration, and mixing duration; f. measuring at least one of the following: encapsulation efficiency, particle size distribution, purification and particle recovery rate, and formulation stability of said LNPs; g. determining the optimal parameters for manufacturing said LNP preparation; and h. manufacturing said LNP preparation based on said optimal parameters.

In various embodiments, the payload is an oligonucleotide. In various embodiments, the oligonucleotide is an antisense molecule. In various embodiments, the oligonucleotide is a siRNA. In various embodiments, the oligonucleotide is a shRNA. In various embodiments, the oligonucleotide is between about 10 to about 30 nucleotides in length. In various embodiments, the payload is an mRNA. In various embodiments, the size of mRNA is about 1 kb to about 2 kb. In various embodiments, the payload is a polypeptide. In various embodiments, said polypeptide is between about 1,000 Da and about 10,000 Da. In various embodiments, the payload is a small molecule. In various embodiments, the small molecule is between about 100 Da and 1000 Da.

In various embodiments, the payload is dissolved in the first solution. In various embodiments, the payload is dissolved in the second solution. In various embodiments, the first solution is an aqueous buffer. In various embodiments, the first solution comprises pH- and osmolality-controlled buffers. In various embodiments, the organic phase of the second solution comprises methanol. In various embodiments, the organic phase of the second solution comprises ethanol.

In various embodiments, the self-assembling molecules include at least a lipid component comprised of at least one species of lipid molecule. In various embodiments, the at least one species of lipid molecule is selected from a cationic lipid species, a non-cationic lipid species, and a phospholipid species. In various embodiments, said second solution comprises more than one type of lipid. In various embodiments, the total concentration of lipid is varied. In various embodiments, the total concentration of lipid is varied between about 0.4 and about 4 mM. In various embodiments, the percentage of lipids that are PEGylated is varied. In various embodiments, the percentage of lipids that are PEGylated are varied between about 0.5% to about 5% of the total lipid composition. In various embodiments, the payload's N:P ratio is varied. In various embodiments, the N:P ratio is varied between about 0.5 to about 5.

In various embodiments, the LNP is a polymer lipid nanoparticle. In various embodiments, the LNP is a liposome. In various embodiments, the LNP is a lipoprotein nanoparticle. In various embodiments, said first solution is injected into said second solution. In various embodiments, said second solution is injected into said first solution. In various embodiments, the optimal parameters are those which produce an encapsulation efficiency of the payload greater than 80%. In various embodiments, the optimal parameters are those which produce a LNP with a mean diameter of 80-200 nm, having a unimodal size distribution, and a polydispersity of less than about 30%. In various embodiments, the LNPs maintain a similar size distribution and payload encapsulation for at least one month under storage in solution at 4 degrees Celsius.

In various embodiments, the present disclosure relates to a workflow for HTS screening of a plurality of parameters for LNP formation, comprising: (i) a robotic liquid handler; (ii) at least one instrument capable of measuring desired LNP characteristics; and (iii) at least one microplate comprising a plurality of microwells; wherein said robotic liquid handler is capable of injecting a plurality of solutions into each of said microwells; wherein said parameters are systematically varied between microwells; and wherein said desired LNP characteristics are capable of being measured for each microwell.

In various embodiments, the plurality of parameters are selected from total lipid content, type of self-assembly molecule; the composition ratio of said self-assembly molecule; the ratio and/or concentration of said self-assembly molecule to said payload; the selection of phase, the buffer type and pH, the injection sequence, volume, and speed, and the mixing duration. In various embodiments, said desired LNP characteristics are selected from the group consisting of: average particle size, particle size distribution, encapsulation efficiency, and particle stability. In various embodiments said instrument is capable of either dynamic light scattering (DLS), ultraviolet-visible (UV-Vis), or fluorescence spectroscopy.

### BRIEF DESCRIPTION OF THE FIGURES

FIGS. 1A-1F show data from the high-speed, ethanol-to-buffer injection followed by multiple rounds of mixing produced uniform LNPs with high ASO loading. LNPs composed of 0.4 μmol of total lipids and 1.5 mol % of DSPE-PEG2000 were mixed with ASO-1 under the N/P ratio of 1 using different mixing conditions. A TECAN® robot was used to investigate reverse injection sequences (ethanol-to-buffer or buffer-to-ethanol) at a speed of 0.1, 0.5, or 0.9 ml/s followed by 10 mixing repeats (FIGS. 1A-1C), or the ethanol-to-buffer injection at a speed of 0.5 or 0.9 ml/s followed by 10 or 20 mixing repeats (FIGS. 1D-1F). Particle size (FIGS. 1A and 1D) and polydispersity (FIGS. 1B and 1E) were measured by dynamic light scattering (DLS). Free ASO-1 was measured by OD260 and calculated for encapsulation efficiency (FIGS. 1C and 1F). Results are mean±SD, n=3; ns, not significant, ****P<0.0001, analyzed by (FIGS. 1A-1C) two-way or one-way (FIGS. 1D-1F) ANOVA followed by Tukey's multiple comparisons.

FIG. 2 shows the HTS workflow for ASO-loaded LNP formulations. Ninety six samples (32 conditions, n=3) varying with 4 levels of lipid composition, 2 levels of total lipid concentration, and 4 levels of ASO loading amount were prepared by the automated solvent-injection method using a TECAN® liquid handler, followed by characterization of particle size distributions by DLS and ASO encapsulation by absorbance at 260 nm. A representative LEA (Laboratory Execution and Analysis) Library Studio design layout was shown for the sample plate.

FIGS. 3A-3E are HTS analyses of ASO-1-loaded LNP formulations. FIG. 3A is an image showing the screening design. Formulation parameters including total lipid concentrations (2 levels), PEGylated lipid contents incorporated in the lipid composition (4 levels), and loading ratios of the ASO (4 levels) were screened in a 96-well plate with 3 replicates for each condition. FIGS. 3B-3D show that samples were diluted in PBS and characterized for particle size distributions by DLS. FIG. 3B is a graph showing representative size distributions, which showed small particle populations with increasing amounts of the PEGylated lipid added in the lipid composition. FIGS. 3C-3D are heat maps showing that LNPs had mean diameters of 45-145 nm and % PD of 10-50%, except large aggregates (diameter of 500-1500 nm) with multimodal size distributions when no DSPE-PEG2000 was incorporated in the lipid composition, as indicated by the “out of range” black spots. Quantitative analyses were also shown for samples with a total lipid concentration of 2 mM. FIG. 3E is a bar graph showing sample aliquots (total lipid concentration of 2 mM) that were measured for the unencapsulated amounts of ASO by OD260 to calculate encapsulation efficiency. Results are mean±SD, n=3; ns, not significant, *P<0.05, **P<0.01, ***P<0.001, and ****P<0.0001, analyzed by two-way ANOVA followed by Tukey's multiple comparisons. This data was confirmed by LC.

FIG. 4 is a bar graph showing that LNPs prepared without the PEGylated lipid produced large aggregates. Mean particle diameters of ASO-1-loaded LNPs prepared without DSPE-PEG2000 (screened conditions shown in the rows A and E in FIGS. 3C-3D) are shown as mean±SD, n=3; ns, not significant, *P<0.05, and ***P<0.001, analyzed by two-way ANOVA followed by Sidak's multiple comparisons.

FIGS. 5A-5C are HTS analyses of ASO-1-loaded, cationic LNP formulations. Screened cationic LNPs showed mean diameters of 60-120 nm (FIG. 5A), polydispersity of 10-50% (FIG. 5B), and similar trends with MC3 LNPs in terms of increasing amounts of the PEGylated lipid. Absence of DSPE-PEG2000 produced large aggregates with multimodal size distributions, as indicated by the “out of range” black spots or incomplete measurements (due to large aggregates) indicated by white spots. Quantitative analyses were also shown for samples with the total lipid concentration of 2 mM. (FIG. 5C) Samples were measured for the amounts of unencapsulated ASO by OD260 to calculate encapsulation efficiency. Results are mean±SD, n=3; ns, not significant, * P<0.05, **P<0.01, ***P<0.001, and ****P<0.0001, analyzed by two-way ANOVA followed by Tukey's (FIGS. 5A-5B) or Sidak's (FIG. 5C) multiple comparisons.

FIGS. 6A-6C are HTS analyses of ASO-2-loaded LNPs formulated with the ionizable lipid under a total lipid concentration of 2 mM, different amounts of DSPE-PEG2000, and different oligonucleotide loading. Results showed similar trends as ASO-1-loaded LNPs (FIGS. 3A-3E) in terms of particle size (FIG. 6A), polydispersity (FIG. 6B), and encapsulation efficiency of the ASO (FIG. 6C). Results are mean±SD, n=3; ns, not significant, *P<0.05, **P<0.01, ***P<0.001, and ****P<0.0001, analyzed by two-way ANOVA followed by Tukey's multiple comparisons.

FIGS. 7A-7E are HTS analyses results that correlated with those from microfluidic preparation using a NanoAssemblr®. FIG. 7A are graphs that show correlations of decreasing particle sizes and increasing polydispersity with increasing amounts of the PEGylated lipid. LNPs were prepared with different molar ratios of DSPE-PEG2000 and a fixed N/P ratio of 2. FIG. 7B is a graph that shows particle sizes were stable under high total lipid concentrations. LNPs were prepared under total lipid concentrations of 0.4, 0.7, 1, or 2 mM, fixed 1.5 mol % of DSPE-PEG2000, and N/P ratio of 2. FIGS. 7C-7D show particle sizes (FIG. 7C) were stable while % EE of ASO (FIG. 7D) decreased under high and excess ASO loading. LNPs were prepared under N/P ratio=5, 2, 1, or 0.5, and 1.5 mol % of DSPE-PEG2000. FIG. 7E. Representative cryo-TEM images of ASO-1-loaded LNPs prepared by the Nanoassemblr® or high-throughput solvent-injection with different formulation parameters. Magnified images showed similar structure patterns of representative LNPs (indicated by blue arrows) prepared with the same formulation parameter using the two approaches. HTS results in the panels (FIGS. 7A, 7C, and 7D) are from the same screening experiment shown in FIG. 3. Results are mean±SD, n=3 except n=1 for microfluidic results in FIG. 7D.

FIGS. 8A-8B show the stability of ASO-1-loaded MC3 LNPs prepared by high-throughput solvent-injection method or NanoAssemblr® at 4° C. for 2 weeks. FIG. 8A is showing mean particle size, and FIG. 8B are graphs showing the polydispersity, over 2 weeks. The total lipid concentration was 2 mM, N/P ratio was 1 (HTS samples) or 0.5 (NanoAssemblr® samples), and PEG amounts varied from 1.5 to 5 mol %. Results are mean±SD, n=3; *P<0.05 and **P<0.01 vs. results at day 0 within each group, analyzed by one-way ANOVA followed by Dunnett's multiple comparisons. Subsequent studies (not shown) have demonstrated similar results after 1 month of storage at 4° C.

FIG. 9 is graphs that show the stability of HTS LNPs shown in FIGS. 8A-8B over 2 weeks at 40° C. Results are mean±SD, n=3; *P<0.05 vs. results at day 0 within each group, analyzed by one-way ANOVA followed by Dunnett's multiple comparisons.

FIG. 10 is a graph showing ASO leakage from LNPs at 40° C. Released ASO-1 from LNPs within 2 weeks was measured by OD260. Results are mean±SD, n=3; *P<0.05 and ns, not significant vs. the 1.5 mol % DSPE-PEG2000 group, analyzed by two-way ANOVA followed by Turkey's multiple comparisons.

FIG. 11 shows that the HTS approach significantly saved raw materials and improved the analytical output compared with microfluidic preparation of ASO-loaded LNPs. Materials needed were calculated for a typical sample with 2 mM total lipids containing 1.5 mol % of DSPE-PEG2000 and the N/P ratio (based on MC3 and ASO-1) of 1.

FIGS. 12A-12B show the alternative method of quantification of ASO encapsulation. FIG. 12A is a schematic of the workflow. ASO-loaded LNPs were prepared by the high-throughput solvent injection method and mixed with the fluorescence probe Sybr-gold, followed by quantification using a fluorescence plate reader (Ex/Em=495/550 nm). FIG. 12B are graphs showing the comparable % encapsulation efficiency for two different LNP formulations prepared under different N/P ratios. Results are mean±SD, n=2; ns, not significant.

FIG. 13A shows the HTS workflow for HiBiT peptide-loaded liposome formulations. Two purification methods, including high-throughput gel filtration and dialysis in 96-well plate formats were compared. LNPs were synthesized by the high-throughput solvent injection method, followed by a characterization of particle size distributions by DLS and free cargo amount by UV-Vis, luminescence, and fluorescence. The LNPs were then purified using either high-throughput gel filtration or dialysis, followed by an analysis of purification efficiency, particle recovery, and particle size stability, using UV-Vis, fluorescence, and DLS, respectively.

FIG. 13B is an image showing the screening design. Formulation parameters including DPPC LNPs without MC3, DPPC LNPs with MC3, DSPC LNPs without MC3, and DSPC LNPs with MC3, with both shielding pegylated lipids and pegylated lipids conjugated with azide were screened in a 96-well plate with 3 replicates for each condition.

FIG. 13C is a heat map showing that LNPs had mean diameters of 50-200 nm, except large aggregates with multimodal size distributions when no DSPE-PEG2000 was incorporated in the lipid composition, as indicated by the “out of range” black spots.

FIGS. 13D-13F are tables showing the quantification of free peptide concentrations before (FIG. 13D) and after purification. Gel filtration and dialysis resulted in mean purification efficiency of ˜98% (FIG. 13E) and ˜61% (FIG. 13F), respectively. A 96-small column plate with MWCO of 40 kD was used for gel filtration and elution with PBS. A 96-well dialysis plate with MWCO of 10 kD was used for dialysis in 3 L PBS overnight, with 3 times of medium change. Loss of data points after dialysis was due to low sample recovery.

FIGS. 13G-13H are data showing the quantification of particle recovery rate and size after purification by gel filtration. FIG. 13G Recovery rates were generally between 80-120%, except for low values due to aggregated samples that were prepared without pegylated lipids. FIG. 13H Particle size distributions remained constant after purification by gel filtration.

FIG. 14 shows a general procedure described herein involving LNPs preparation via automated solvent injection using a robotic liquid handler (top left) and high-throughput screening (bottom left), followed by analysis for quality attributes, e.g. particle size distribution (top right), ASO encapsulation (bottom middle) and LNP stability (bottom right).

FIGS. 15A-15E are schematic representation of the ASO-LNP formulation library. A liquid handling robot was used to formulate ASO-LNPs by rapidly mixing an aqueous phase containing the ASO with an ethanol phase containing the dissolved lipid mixture with varying PEG-lipid compositions (FIG. 15A). Each lipid mixture comprised a distinct PEG-lipid selected from the phosphoglyceride, diglyceride, or ceramide (FIG. 15B) families, in combination with the ionizable lipid MC3 (FIG. 15C), cholesterol (FIG. 15D), and helper lipid DSPC (FIG. 15E) to generate the ASO-LNP library with 54 different formulations.

FIGS. 16A-16F show particle size distribution of ASO-LNPs. ASO-LNPs were prepared with varying types and amounts of PEGylated lipids using a liquid handling robot. The PEG-lipid analog used in the respective ASO-LNP formulation is depicted by the #underlying the X-axis labels and can be referenced from Table 1. The particle library was characterized using dynamic light scattering in a 96-well plate setup to identify particle size (FIGS. 16A-16C) and polydispersity trends (FIGS. 16D-16F) across different subsets of anionic (linear, branched) and neutral PEG-lipids, as a function of the PEG size (Da) indicated by the X-axis values, C-tail type indicated by the different colored backgrounds, as well as the PEG-lipid content in the ASO-LNP (mol %) indicated by the respective bar colors.

FIG. 17 shows behavioral trends across the ASO-LNP HTS library. Mean particle diameters in FIGS. 16A-16C are represented using a color-coded heatmap.

FIG. 18 shows translations of hit ASO-LNPs to scaled-up formulations. ASO-LNP formulations were identified from the 1, 3, and 5 mol % data in our HTS library and were scaled-up using a microfluidic mixer. A smooth translation across the two formulation scales and techniques was validated by comparing their size distributions.

### DETAILED DESCRIPTION

Lipid nanoparticle (LNP) manufacturing for drug delivery is challenging due to their complicated physicochemical properties that are affected by various formulation parameters. Controlling for particle structure and size distribution, physicochemical properties of the particle surface, lipid content, amount of the free API and encapsulation efficiency, and physical and chemical stability in LNP manufacture is difficult and complicated. Screening of LNP formulation parameters, including lipid species, percentage, concentration and drug loading, by conventional batch methods requires significant time and raw materials. Therefore, a high-throughput screening approach with minimal material inputs and efficient preparation and analytical outputs would be preferred to determine a lead formulation candidate with optimal quality attributes. Robotic liquid handlers are mainly used for liquid addition and transfer and have not been used as a LNP formulator with fine-tuned instrument parameters. Further, there is a lack of streamlined high-throughput workflow integrating both LNP preparation and analytics. Provided herein are high-throughput methods for optimizing LNP manufacture based on desired characteristics using a robotic liquid handler for injection-based LNP formation. Further provided herein are optimized LNP particles and methods of their manufacturer.

It will be understood that descriptions herein are exemplary and explanatory only and are not restrictive of the invention as claimed. In this application, the use of the singular includes the plural unless specifically stated otherwise.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. As utilized in accordance with the present disclosure, the following terms, unless otherwise indicated, shall be understood to have the following meanings:

In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

The term “polynucleotide”, “nucleotide”, or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′, 3′-dideoxyribose, and internucleotide linkage modifications such as phosphorothioate, phosphorodithioate, phosphoroselenoate, phosphoro-diselenoate, phosphoro-anilothioate, phoshoraniladate and phosphoroamidate.

The term “oligonucleotide” refers to a polynucleotide comprising 200 or fewer nucleotides. Oligonucleotides can be single stranded or double stranded, e.g., for use in the construction of a mutant gene. Oligonucleotides can be sense or antisense oligonucleotides. An oligonucleotide can include a label, including a radiolabel, a fluorescent label, a hapten or an antigenic label, for detection assays. Oligonucleotides can be used, for example, as PCR primers, cloning primers or hybridization probes.

The terms “polypeptide” or “protein” refer to a macromolecule having the amino acid sequence of a protein, including deletions from, additions to, and/or substitutions of one or more amino acids of the native sequence. The terms “polypeptide” and “protein” specifically encompass antigen-binding molecules, antibodies, or sequences that have deletions from, additions to, and/or substitutions of one or more amino acid of antigen-binding protein. The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. Useful polypeptide fragments include immunologically functional fragments of antigen-binding molecules.

The term “isolated” means (i) free of at least some other proteins with which it would normally be found, (ii) is essentially free of other proteins from the same source, e.g., from the same species, (iii) separated from at least about 50 percent of polynucleotides, lipids, carbohydrates, or other materials with which it is associated in nature, (iv) operably associated (by covalent or noncovalent interaction) with a polypeptide with which it is not associated in nature, or (v) does not occur in nature.

A “variant” of a polypeptide (e.g., an antigen-binding molecule) comprises an amino acid sequence wherein one or more amino acid residues are inserted into, deleted from and/or substituted into the amino acid sequence relative to another polypeptide sequence. Variants include, e.g., fusion proteins.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”).

To calculate percent identity, the sequences being compared are typically aligned in a way that gives the largest match between the sequences. One example of a computer program that can be used to determine percent identity is the GCG program package, which includes GAP (Devereux et al., Nucl. Acid Res., 1984, 12, 387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). In certain embodiments, a standard comparison matrix (see, e.g., Dayhoff et al., 1978, Atlas of Protein Sequence and Structure, 5:345-352 for the PAM 250 comparison matrix; Henikoff et al., 1992, Proc. Natl. Acad. Sci. U.S.A., 89, 10915-10919 for the BLO-SUM 62 comparison matrix) is also used by the algorithm.

The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified antigen-binding molecule can have a greater circulating half-life than an antigen-binding molecule that is not chemically modified. In some embodiments, a derivative antigen-binding molecule is covalently modified to include one or more water soluble polymer attachments, including, but not limited to, polyethylene glycol, polyoxyethylene glycol, or polypropylene glycol.

Peptide analogs are commonly used in the pharmaceutical industry as non-peptide drugs with properties analogous to those of the template peptide. These types of non-peptide compound are termed “peptide mimetics” or “peptidomimetics.” Fauchere, J. L., 1986, Adv. Drug Res., 1986, 15, 29; Veber, D. F. & Freidinger, R. M., 1985, Trends in Neuroscience, 8, 392-396; and Evans, B. E., et al., 1987, J. Med. Chem., 30, 1229-1239, which are incorporated herein by reference for any purpose.

The term “therapeutically effective amount” refers to the amount of immune cells or other therapeutic agent determined to produce a therapeutic response in a mammal. Such therapeutically effective amounts are readily ascertained by one of ordinary skill in the art.

The terms “patient” and “subject” are used interchangeably and include human and non-human animal subjects as well as those with formally diagnosed disorders, those without formally recognized disorders, those receiving medical attention, those at risk of developing the disorders, etc.

The term “treat” and “treatment” includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. The term “prevent” does not require the 100% elimination of the possibility of an event. Rather, it denotes that the likelihood of the occurrence of the event has been reduced in the presence of the compound or method.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% higher compared to a reference quantity, level, value, number, frequency, percentage, dimension, size, amount, weight or length. In one embodiment, the terms “essentially the same” or “substantially the same” refer to a range of quantity, level, value, number, frequency, percentage, dimension, size, amount, weight or length that is about the same as a reference quantity, level, value, number, frequency, percentage, dimension, size, amount, weight or length.

As used herein, the terms “substantially free of” and “essentially free of” are used interchangeably, and when used to describe a composition, such as a cell population or culture media, refer to a composition that is free of a specified substance, such as, 95% free, 96% free, 97% free, 98% free, 99% free of the specified substance, or is undetectable as measured by conventional means. Similar meaning can be applied to the term “absence of,” where referring to the absence of a particular substance or component of a composition.

As used herein, the term “appreciable” refers to a range of quantity, level, value, number, frequency, percentage, dimension, size, amount, weight or length or an event that is readily detectable by one or more standard methods. The terms “not-appreciable” and “not appreciable” and equivalents refer to a range of quantity, level, value, number, frequency, percentage, dimension, size, amount, weight or length or an event that is not readily detectable or undetectable by standard methods. In one embodiment, an event is not appreciable if it occurs less than 5%, 4%, 3%, 2%, 1%, 0.1%, 0.001%, or less of the time.

Throughout this specification, unless the context requires otherwise, the words “comprise,” “comprises” and “comprising” will be understood to imply the inclusion of stated step or element or group of steps or elements but not the exclusion of any other step or element or group of steps or elements. In particular embodiments, the terms “include,” “has,” “contains,” and “comprise” are used synonymously.

As used herein, “consisting of” is meant including, and limited to, whatever follows the phrase “consisting of”. Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” or “approximately” refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5% or 1%, or any intervening ranges thereof.

### High Throughput Screening Methods for Optimizing Manufacture of Lipid Nanoparticles

To address the needs of screening and optimization of lipid-based nanomedicines, the disclosure herein provides for a high-throughput screening (HTS) workflow for the preparation of lipid nanoparticles, and for the characterization of their particle size distributions and payload encapsulation.

In various embodiments, the present disclosure relates to high throughput screening methods for optimizing the manufacture of lipid nanoparticles (LNPs). In various embodiments, the methods disclosed herein utilize a high throughput screening (HTS) screening workflow including (i) a robotic liquid handler, (ii) at least one instrument capable of measuring desired LNP characteristics; and (iii) at least one microplate, wherein said microplate comprises a plurality of microwells. In various embodiments, LNPs are formed through the HTS screening workflow described above using the solvent-injection method. See, e.g., Gentine et al., 2012, J Liposome Res. 22, 18-30; Schubert and Muller-Goymann, 2003, Eur. J. Pharm. Biopharm. 55, 125-131.

In various embodiments, the HTS workflow includes an instrument capable of measuring desired LNP characteristics. Such characteristics include, encapsulation efficiency, mean particle size, and particle size distributions. Physical stability can also be determined by measuring particle size and payload release at different time points after storage. Such analytical techniques are known in the art, and include scanning/transmission electron microscopy (SEM/TEM), atomic force microscopy (AFM) analytical ultracentrifugation (AUC), dynamic light scattering (DLS), ultraviolet (UV) spectroscopy, and flow field fractionation (FFF). In various embodiments, the HTS workflow includes an instrument capable of DLS, UV-Vis, or fluorescence spectroscopy. In various embodiments, the methods disclosed herein utilize a high throughput screening (HTS) screening workflow including (i) a robotic liquid handler, (ii) an instrument capable of performing DLS; (iii) an instrument capable of UV-Vis or fluorescence spectroscopy on a sample; and (iv) at least one microplate, wherein said microplate comprises a plurality of microwells.

In various embodiments, the HTS workflow provides for a method of optimizing LNP manufacturing using a solvent-injection system. As used herein, a “solvent-injection system” means rapidly injecting a first solution comprising lipid-comprising self-assembling molecules into a second solution. In various embodiments, the solutions are intermixable or miscible. In various embodiments, the first solution is a water-miscible solvent. In various embodiments, at least one solution is an organic phase solvent. Acetone, ethanol, isopropanol and methanol are all suitable solvents for LNP preparation. In various embodiments, the first solution is an alcohol. In various embodiments, the first solution is ethanol. In various embodiments the first solution is methanol.

In various embodiments, the payload to be encapsulated by the LNP is dissolved in said second solution. In various embodiments, the payload to be encapsulated by the LNP is dissolved in said first solution. In various embodiments, the payload is encapsulated by a third water-miscible solvent.

In various embodiments, at least two of the solutions are different phases. In various embodiments there are three solutions injected into one another. In various embodiments there are at least four solutions injected into one another. In various embodiments there is at least one organic phase and at least one aqueous phase.

In various embodiments, one the solutions comprises an aqueous solvent. In various embodiments the aqueous solvent is an aqueous buffer.

The injection of one solution into another is controlled by a robotic liquid handler. As used herein, the term “robotic liquid handler” means a device capable of automatically pipetting, transferring and mixing liquids into a plurality of wells, microwells or other liquid reservoir in parallel. In various embodiments, the robotic liquid handler is capable of delivering liquids of different composition or different amounts to different wells, microwells or liquid reservoir in parallel. In various embodiments, the robotic liquid handler is capable of pipetting, transferring and mixing liquids to different wells, microwells or liquid reservoirs in parallel at varying speeds or durations.

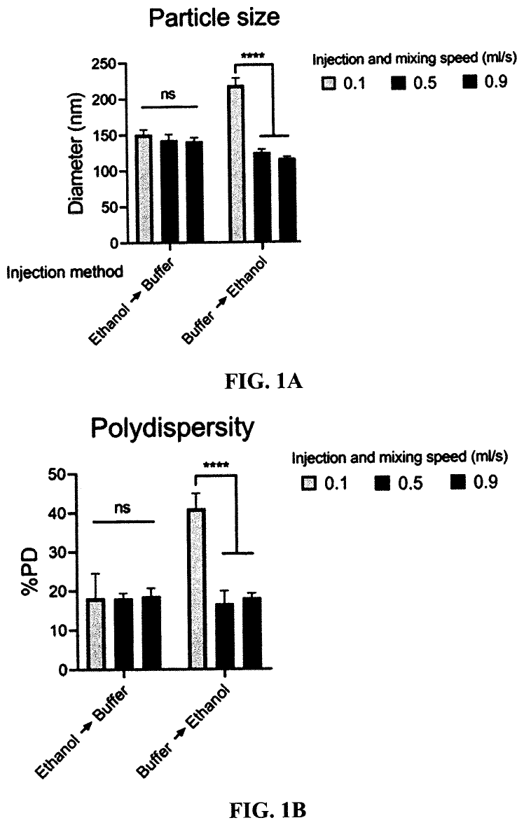

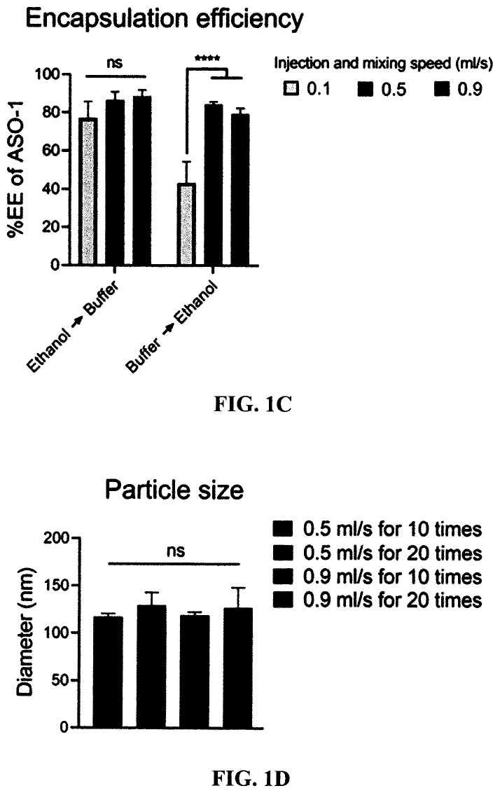

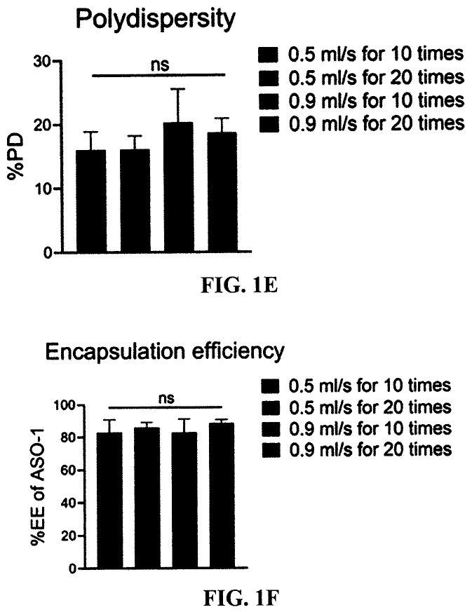

In various embodiments, after injecting said one solution into said second solution, the robotic liquid handler repeatedly takes up and re-injects said solutions, thereby mixing the at least two solutions. In various embodiments, the speed and duration of this injection and/or mixing is varied to determine the optimal parameters for LNP formation. In various embodiments, the speed of injection and/or mixing is varied from 0.1 ml/s to 0.9 ml/s. In various embodiments, the initial injection speed (i.e. the first injection of liquid) is performed at a speed of 0.1 ml/s to 0.9 ml/s. See FIG. 1. In various embodiments, the subsequent injections/mixing is performed over 1-10 s (10× mix at 0.1 ml/s to 0.9 ml/s).

In various embodiments, the LNP formation is completed in at least one microplate. In various embodiments, the microplate is comprised of a plurality of microwells, wherein the formation conditions (e.g. lipid species, lipid composition, total lipid concentration, payload, payload loading ratio, phase species) are varied between microwells. The microplate can be of any size and comprise any number of microwells. In various embodiments, the microplate comprises 4, 6, 8, 12, 24, 48, 96, 384, 1536 microwells.

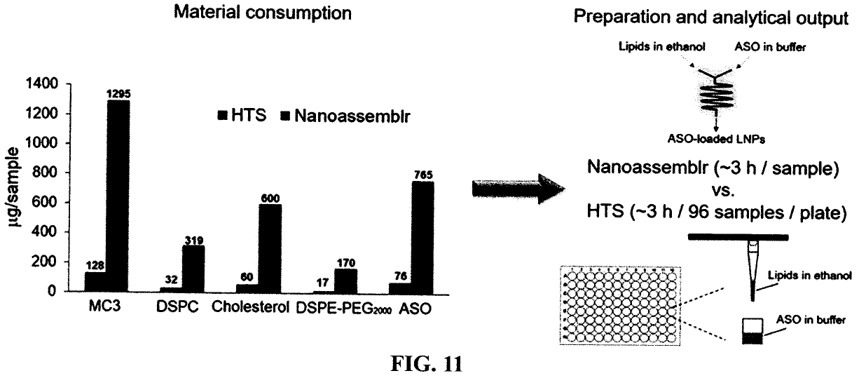

One advantage of the HTS methods provided herein, is that LNP formation can occur rapidly in a small amount of solution. The methods disclosed herein decrease material consumption by 10 fold, and improve processing outputs by 100 fold (see FIG. 11). LNP formation in microwells use considerably less material than LNPs formed for example using a microfluidic-based preparation. In various embodiments, the microwell is about 10 μL, about 20 μL, about 30 μL, about 40 μL, about 50 μL, about 60 μL, about 70 μL, about 80 μL, about 90 μL, about 100 μL, about 125 μL, about 150 μL, about 175 μL, about 200 μL, about 250 μL, about 350 μL, about 360 L, about 400 μL, about 500 μL, about 1000 μL, about 2000 μL, about 3000 μL, about 4000 μL in volume.

### Lipid Nanoparticles (LNPs)

Provided herein are optimized lipid nanoparticles, as well as methods for optimizing the manufacture of these lipid nanoparticles “LNPs”. As used herein, the term “lipid nanoparticle” or “LNP” refers to a composition including (i) a plurality of self-assembling molecules, wherein said self-assembling molecules include a lipid component; and (ii) a payload. The LNPs whose manufacture is optimized using the present invention can be used for any purpose. In various embodiments the optimized LNPs may be used to deliver a vaccine. In various embodiments, the optimized LNPs may be used deliver a drug to patient in need thereof. The LNP may carry any payload, including but not limited to nucleic acids, peptides, proteins and small molecules. Further, the LNP may consist solely of lipids (for example a liposome) or may include other components such as polymers or proteins capable of self-assembly.

In various embodiments the LNP is an optimized LNP manufactured using the techniques described above. In various embodiments, the optimized LNP is manufactured by a process comprising the steps of (i) obtaining a first solution comprising an aqueous phase, (ii) obtaining a second solution comprising an organic phase and a plurality of molecules capable of self-assembly, and wherein said first and second solutions are intermixable; (iii) dissolving at least one payload molecule into either the first or second solution; (iv) using a robotic liquid handler to prepare and dispense said phases with varied compositions into a plurality of wells; (v) mixing said first and second solutions to obtain lipid nanoparticles encapsulating said payload using said robotic handler under conditions suitable for LNP formation, wherein at least one of the following conditions are varied amongst different wells: type of self-assembly molecule, composition ratio of said self-assembly molecule; ratio and/or concentration of said self-assembly molecule to said payload, the selection of phase, buffer type and pH, the injection sequence, injection speed, mixing speed, volume, phase ratio, injection duration, and mixing duration; (vi) measuring at least one of the following: encapsulation efficiency, particle size distribution, purification and particle recovery rate, and formulation stability of said LNPs; (vii) determining the optimal parameters for manufacturing said LNP preparation; and (viii) manufacturing said LNP preparation based on said optimal parameters.

In various embodiments, the present invention relates to methods of manufacturing LNPs using high throughput methods, comprising the steps of (i) obtaining a first solution comprising an aqueous phase, (ii) obtaining a second solution comprising an organic phase and a plurality of molecules capable of self-assembly, and wherein said first and second solutions are intermixable; (iii) dissolving at least one payload molecule into either the first or second solution; (iv) using a robotic liquid handler to prepare and dispense said phases with varied compositions into a plurality of wells; (v) mixing said first and second solutions to obtain lipid nanoparticles encapsulating said payload using said robotic handler under conditions suitable for LNP formation.

### Self-Assembling Molecules

As used herein, the term “self-assembling molecule”, refers to any molecule capable of a defined arrangement without guidance or management from an outside source. The optimized LNPs may be comprised of a single species of self-assembling molecule or may be comprised of a plurality of species of self-assembling molecule. In various embodiments, the optimized LNPs include a lipid-component with at least one species of lipid molecule. In various embodiments, the LNP may include a polymer molecule and/or a protein/peptide molecule. In various embodiments, the self-assembling molecules of the LNP may only include lipid molecules.

The lipid component may comprise a single lipid species, or it may include more than one type of lipid. In various embodiments of the present invention, the relative composition of lipid in a LNP preparation will be varied. In various embodiments different species of lipids or different combinations of lipid species will be evaluated when considering the optimal parameters for manufacture of a given LNP formulation. In various embodiments, at least one lipid molecule is pegylated. In various embodiments, the lipid component may include phospholipids.

In various embodiments, the LNP formulation may comprise one or more cationic or ionizable lipids. In some embodiments, the one or more cationic lipids are selected from the group consisting of cKK-E12, OF-02, C12-200, MC3, DLinDMA, DLinkC2DMA, ICE (Imidazol-based), HGT5000, HGT5001, HGT4003, DODAC, DDAB, DMRIE, DOSPA, DOGS, DODAP, DODMA and DMDMA, DODAC, DLenDMA, DMRIE, CLinDMA, CpLinDMA, DMOBA, DOcarbDAP, DLinDAP, DLincarbDAP, DLinCDAP, KLin-K-DMA, DLin-K-XTC2-DMA, 3-(4-(bis(2-hydroxydodecyl)amino)butyl)-6-(4-((2-hydroxydodecyl)(2-hydroxyundecyl)amino)butyl)-1,4-dioxane-2,5-dione (Target 23), 3-(5-(bis(2-hydroxydodecyl)amino)pentan-2-yl)-6-(5-((2-hydroxydodecyl)(2-hydroxyundecyl)amino)pentan-2-yl)-1,4-dioxane-2,5-dione (Target 24), N1GL, N2GL, V1GL and combinations thereof.

In some embodiments, the one or more cationic or ionizable lipids are amino lipids. In various embodiments, the amino lipids are primary, secondary, tertiary, quaternary amines, pyrroldine or piperdine. Amino lipids suitable for use in the invention include those described in WO2017180917, which is hereby incorporated by reference. Exemplary aminolipids in WO2017180917 include those described at paragraph [0744] such as DLin-MC3-DMA (MC3), (13Z,16Z)—N,N-dimethyl-3-nonyldocosa-13,16-dien-1-amine (L608), and Compound 18. Other amino lipids include Compound 2, Compound 23, Compound 27, Compound 10, and Compound 20. Further amino lipids suitable for use in the invention include those described in WO2017112865, which is hereby incorporated by reference. Exemplary amino lipids in WO2017112865 include a compound according to one of formulae (I), (Ia1)-(Ia6), (lb), (II), (Ila), (III), (Ilia), (IV), (17-1), (19-1), (19-11), and (20-1), and compounds of paragraphs [00185], [00201], [0276]. In some embodiments, cationic lipids suitable for use in the invention include those described in WO2016118725, which is hereby incorporated by reference. Exemplary cationic lipids in WO2016118725 include those such as KL22 and KL25. In some embodiments, cationic lipids suitable for use in the invention include those described in WO2016118724, which is hereby incorporated by reference. Exemplary cationic lipids in WO2016118725 include those such as KL10, 1,2-dilinoleyloxy-N,N-dimethylaminopropane (DLin-DMA), and KL25.

In some embodiments, the LNP formulation will comprise one or more non-cationic lipids. In some embodiments, the one or more non-cationic lipids are selected from DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DOPC (1,2-dioleyl-sn-glycero-3-phosphotidylcholine) DPPE (1,2-dipalmitoyl-sn-glycero-3-phosphoethanolamine), DMPE (1,2-dimyristoyl-sn-glycero-3-phosphoethanolamine), DOPG (1,2-dioleoyl-sn-glycero-3-phospho-(1′-rac-glycerol)).

In some embodiments, the LNP formulation comprises one or more PEG-modified lipids. In some embodiments, the one or more PEG-modified lipids comprise a poly(ethylene) glycol chain of up to 5 kDa in length covalently attached to a lipid with alkyl chain(s) of C₆-C₂₀ length. A PEG lipid may be selected from the non-limiting group consisting of PEG-modified phosphatidyletanolamines, PEG-modified phosphatidic acids, PEG-modified ceramides, PEG-modified dialkylamines, PEG-modified diacylgycerols, nad PEG-modified dialkylglycerols. For example, a PEG lipid may be PEG-c-DOMG, PEG-DMG, PEG-DLPE, PEG-DMPE, PEG-DPPC or a PEG-DSPE lipid.

In various embodiments, the percentage of lipids that are PEGylated (i.e. PEG density) within the LNP are varied. Polyethylene glycol (PEG) density in the LNP has been found to impact particle size, surface charge and stability. In various embodiments, the PEG density is varied between about 0.1% and about 10%. In various embodiments, the PEG density is varied between about 0.2% and about 9%. In various embodiments, the PEG density is varied between about 0.3% and about 8%. In various embodiments, the PEG density is varied between about 0.4% and about 7%. In various embodiments, the PEG density is varied between about 0.5% and about 6%. In various embodiments, the PEG density is varied between about 0.5% and about 5%.

In various embodiments, the total concentration of the lipid component present in the solution for LNP preparation is varied in order to achieve the optimal characteristics for any given LNP. In various embodiments, the total concentration of lipid is varied between about 0.1 mM and about 8 mM. In various embodiments, the total concentration of lipid is varied between about 0.2 mM and about 7 mM. In various embodiments, the total concentration of lipid is varied between about 0.3 mM and about 6 mM. In various embodiments, the total concentration of lipid is varied between about 0.4 mM and about 4 mM. In various embodiments, the total concentration of lipid is varied between about 0.5 mM and about 3 mM.

In various embodiments, the LNP will comprise more than one type or species of lipid. In various embodiments, the LNP will comprise at least 2 types of lipids. In various embodiments, the LNP will comprise at least 3 types of lipids. In various embodiments, the LNP will comprise at least 4 types of lipids. In various embodiments, the LNP will comprise at least 5 types of lipids. In various embodiments, the LNP will comprise at least 6 types of lipids. In various embodiments, the LNP will comprise at least 7 types of lipids.

The lipid component of a nanoparticle composition may include one or more structural lipids. The nanoparticle compositions of the present invention may include a structural lipid (e.g., cholesterol fecosterol, sitosterol, campesterol, stigmasterol, brassicasterol, ergosterol, tomatidine, tomatine, ursolic acid, or alpha-tocopherol).

The lipid component of a nanoparticle composition may include one or more phospholipids, such as one or more (poly)unsaturated lipids. In general, such lipids may include a phospholipid moiety and one or more fatty acid moieties.

A phospholipid moiety may be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated.

In some embodiments a nanoparticle composition may include 1,2-distearoyl-sn-glycero-3-phosphocholine (DSPC), 1,2-dioleoyl-sn-glycero-3-phosphoethanolamine (DOPE), or both DSPC and DOPE. Phospholipids useful in the compositions and methods of the invention may be selected from the non-limiting group consisting of DSPC, DOPE, 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

The LNP composition may include one or more components in addition to those described in the preceding sections. For example, a nanoparticle composition may include one or more small hydrophobic molecules such as a vitamin (e.g., vitamin A or vitamin E) or a sterol.

LNP compositions may also include one or more permeability enhancer molecules, carbohydrates, polymers, therapeutic agents, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a LNP composition. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellu lose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, polyoxamines, poly(ortho)esters, poly(butyric acid), poly(valeric acid), poly(lactide-co-caprolactone), and trimethylene carbonate, polyvinylpyrrolidone.

Therapeutic agents may include, but are not limited to, cytotoxic, chemotherapeutic, and other therapeutic agents. Cytotoxic agents may include, for example, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, rachelmycin, and analogs thereof. Radioactive ions may also be used as therapeutic agents and may include, for example, radioactive iodine, strontium, phosphorous, palladium, cesium, iridium, cobalt, yttrium, samarium, and praseodymium. Other therapeutic agents may include, for example, antimetabolites (e.g., methotrexate, 6-mercaptopurine, 6-thioguanine, cytarabine, and 5-fluorouracil, and decarbazine), alkylating agents (e.g., mechlorethamine, thiotepa, chlorambucil, rachelmycin, melphalan, carmustine, lomustine, cyclophosphamide, busulfan, dibromomannitol, streptozotocin, mitomycin C, and cis-dichlorodiamine platinum (II) (DDP), and cisplatin), anthracyclines (e.g., daunorubicin and doxorubicin), antibiotics (e.g., dactinomycin, bleomycin, mithramycin, and anthramycin), and anti-mitotic agents (e.g., vincristine, vinblastine, taxol, and maytansinoids).

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin 134, dornase alfa, neltenexine, and erdosteine), and DNases (e.g., rhDNase). A surface altering agent may be disposed within a LNP and/or on the surface of a LNP composition (e.g., by coating, adsorption, covalent linkage, or other process).

In addition to these components, LNP compositions of the invention may include any substance useful in pharmaceutical compositions. For example, the LNP composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see, e.g., Remington's The Science and Practice of Pharmacy, 21st Edition, A. R. Gennaro; Lippincott, Williams & Wilkins, Baltimore, Md., 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent may be starch (e.g. cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Preservatives include, but are not limited to, antioxidants, chelating agents, antimicrobial preservatives, antifungal preservatives, alcohol preservatives, acidic preservatives, and/or other preservatives. Antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, phenol, benzyl alcohol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g. HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

In various embodiments, the LNP may be a liposome. In various embodiments, the LNP may be polymer-lipid nanoparticle. In various embodiments, the LNP may include additional protein or peptide molecules.

### Payloads

The LNPs of the present invention are manufactured to encapsulate a payload. The term “payload” refers to any chemical entity, pharmaceutical, drug (such drug can be, but not limited to, a small molecule, an inorganic solid, a polymer, or a biopolymer), small molecule, nucleic acid (e.g., DNA, RNA, siRNA, etc.), protein, peptide and the like that is complexed with a lipid nanoparticle formulation described in the present disclosure. A payload also encompasses a candidate (e.g., of unknown structure and/or function) for sue to treat or prevent a disease, illness, sickness, or disorder of bodily function and includes, but is not limited to, test compounds that are both known and potential therapeutic compounds. A test compound can be determined to be therapeutic by screening using the screening methods of the present disclosure.

In various embodiments, the payload is comprised of one or more nucleotides. For example, in various embodiments, the payload is an oligonucleotide. In various embodiments, such payload encapsulated LNPs may be characterized by an N:P ratio. As used herein, “N/P ratio” refers to the ratio of positively-chargeable polymer amine (N=nitrogen) groups to negatively-charged nucleic acid phosphate (P) groups. The N/P ratio plays an important role in intracellular payload delivery. In various embodiments, the payload's N:P ratio is varied. In various embodiments, the N:P ratio is varied between about 0.5 to about 5. In various embodiments, the N:P ratio is varied between about 0.25 and about 10. In various embodiments, the N:P ratio is about 0.1, about 0.2, about 0.25, about 0.5, about 1, about 1.5, about 2, about 2.5, about 3, about 3.5, about 4, about 4.5, about 5, about 6, about 7, about 8 about 9, or about 10.

In various embodiments, the payload is an oligonucleotide. In various embodiments, the oligonucleotide is an antisense molecule. In various embodiments, the oligonucleotide is an siRNA. In various embodiments, the oligonucleotide is an shRNA. The oligonucleotide may be of a varied length. In various embodiments, the oligonucleotide is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 nucleotides in length. In various embodiments, the oligonucleotide is between about 2 and about 40 nucleotides in length. In various embodiments, the oligonucleotide is between about 4 and about 35 nucleotides in length. In various embodiments, the oligonucleotide is about 10 and about 30 nucleotides in length. In various embodiments, the oligonucleotide is between about 12 and about 17 nucleotides in length.

In various embodiments, the payload is an mRNA. In various embodiments, that mRNA is about 500-3000 nucleotides in length. In various embodiments, the mRNA is 500 nucleotides, 1000 nucleotides, 1500 nucleotides, 2000 nucleotides, 2500 nucleotides, 3000 nucleotides in length. In various embodiments, the mRNA encodes an antigenic peptide. In various embodiments, the mRNA is part of a vaccine.

In various embodiments, the payload is a polypeptide. In various embodiments, the polypeptide is between about 1,000 and 10,000 Da. In various embodiments, the polypeptide is about 500 Da, about 600 Da, about 700 Da, about 800 Da, about 900 Da, about 1,000 Da, about 1,500 Da, about 2,000 Da, about 2,500 Da, about 3,000 Da, about 3,500 Da, about 4,000 Da, about 4,500 Da, about 5,000 Da, about 5,500 Da, about 6,000 Da, about 6,500 Da, about 7,000 Da, about 7,500 Da, about 8,000 Da, about 8,500 Da, about 9,000 Da, about 9,500 Da, about 10,000 Da, about 15,000 Da or about 20,000 Da.

In various embodiments, the payload is a small molecule. In various embodiments, the small molecule is between about 100 Da and 1000 Da. In various embodiments, the small molecule is about 50 Da, about 60 Da, about 70 Da, about 80 Da, about 90 Da, about 100 Da, about 150 Da, about 200 Da, about 250 Da, about 300 Da, about 350 Da, about 400 Da, about 450 Da, about 500 Da, about 550 Da, about 600 Da, about 650 Da, about 700 Da, about 750 Da, about 800 Da, about 850 Da, about 900 Da, about 950 Da, about 1,000 Da, about 1,500 Da or about 2,000 Da.

### Pharmaceutical Preparations

In various embodiments, the optimized lipid nanoparticle may be formulated in whole or in part as a pharmaceutical preparation. Pharmaceutical preparation of the invention may include one or more nanoparticle compositions. For example, a pharmaceutical composition may include one or more nanoparticle compositions including one or more different payloads. Pharmaceutical compositions of the invention may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21st Edition, A. R. Gennaro; Lippincott, Williams & Wilkins, Baltimore, Md., 2006. Conventional excipients and accessory ingredients may be used in any pharmaceutical composition of the invention, except insofar as any conventional excipient or accessory ingredient may be incompatible with one or more components of a nanoparticle composition of the invention. An excipient or accessory ingredient may be incompatible with a component of a nanoparticle composition if its combination with the component may result in any undesirable biological effect or otherwise deleterious effect.

In some embodiments, one or more excipients or accessory ingredients may make up greater than 50% of the total mass or volume of a pharmaceutical composition including a nanoparticle composition of the invention. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more nanoparticle compositions, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more nanoparticle compositions.

Nanoparticle compositions and/or pharmaceutical compositions including one or more nanoparticle compositions may be administered to any patient or subject, including those patients or subjects that may benefit from a therapeutic effect provided by the delivery of an mRNA to one or more particular cells, tissues, organs, or systems or groups thereof, such as the renal system. Although the descriptions provided herein of nanoparticle compositions and pharmaceutical compositions including nanoparticle compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition including one or more nanoparticle compositions may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., nanoparticle composition). The amount of the active ingredient is generally equal to the dosage of the active ingredient which would be administered to a subject and/or a convenient fraction of such a dosage such as, for example, one-half or one-third of such a dosage.

Pharmaceutical compositions of the invention may be prepared in a variety of forms suitable for a variety of routes and methods of administration. For example, pharmaceutical compositions of the invention may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, USP, and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, films, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay, silicates), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a composition may include ointments, pastes, creams, lotions, gels, powders, solutions, sprays, inhalants, and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (wt/wt) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (wt/wt) of the composition, and active ingredient may constitute 0.1% to 20% (wt/wt) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 m to 500 m. Such a formulation is administered in the manner in which snuff is taken, i.e. by rapid inhalation through the nasal passage from a container of the powder held close to the nose.

Formulations suitable for nasal administration may, for example, comprise from about as little as 0.1% (wt/wt) and as much as 100% (wt/wt) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (wt/wt) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (wt/wt) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this present disclosure.

A nanoparticle composition including one or more payloads may be administered by any route. In some embodiments, compositions of the invention, including prophylactic, diagnostic, or imaging compositions including one or more nanoparticle compositions of the invention, are administered by one or more of a variety of routes, including oral, intravenous, intramuscular, intra-arterial, intramedullary, intrathecal, subcutaneous, intraventricular, trans- or intra-dermal, interdermal, rectal, intravaginal, intraperitoneal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, or subcutaneously. However, the present disclosure encompasses the delivery of compositions of the invention by any appropriate route taking into consideration likely advances in the sciences of drug delivery. In general, the most appropriate route of administration will depend upon a variety of factors including the nature of the nanoparticle composition including one or more mRNAs (e.g., its stability in various bodily environments such as the bloodstream and gastrointestinal tract), the condition of the patient (e.g., whether the patient is able to tolerate particular routes of administration), etc.

In certain embodiments, compositions in accordance with the present disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.1 mg/kg to about 10 mg/kg, from about 1 mg/kg to about 5 mg/kg, from about 2 mg/kg to about 5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, or from about 0.1 mg/kg to about 1 mg/kg in a given dose, where a dose of 1 mg/kg provides 1 mg of a composition per 1 kg of subject body weight. In particular embodiments, a dose of about 0.005 mg/kg to about 5 mg/kg of a nanoparticle composition of the invention may be administrated. A dose may be administered one or more times per day, in the same or a different amount, to obtain a desired level of mRNA expression and/or therapeutic, diagnostic, prophylactic, or imaging effect. The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In some embodiments, a single dose may be administered, for example, prior to or after a surgical procedure or in the instance of an acute disease, disorder, or condition.

Nanoparticle compositions including one or more payloads may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. For example, one or more nanoparticle compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the invention, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

### EXAMPLES

The following examples are not meant to be limiting, but are presented to provide further information and support for the present invention. The Examples below demonstrate that the HTS method of optimizing LNP formation for optimal payload loading and particle size distribution can be directly translated to scaled up manufacturing processes such as microfluidic-based approaches. This HTS approach decreased material consumption by ˜10 folds and improved processing outputs by ˜100 folds. These results indicate the robustness and utility of the HTS methods for optimizing LNP manufacturing, therefore promoting their clinical translation.

### Materials and Methods

### Materials

Lipids including 1,2-distearoyl-sn-glycero-3-phosphocholine (DSPC), 1,2-distearoyl-sn-glycero-3-phosphoethanolamine-N-[methoxy(polyethylene glycol)-2000] (DSPE-PEG₂₀₀₀), and the cationic 1,2-dioleoyl-3-trimethylammonium-propane (DOTAP) were purchased from Avanti Polar Lipids (AL, USA). The ionizable lipid dilinoleylmethyl-4-dimethylaminobutyrate (DLin-MC3-DMA, MC3) was from MCE (NJ, USA), and cholesterol was from Sigma (MO, USA). Two model ASOs, ASO-1 (13-mer, Na-salt form) and ASO-2 (16-mer, Na-salt form) were synthesized in house. All other reagents were at least reagent grade and DNase/RNAse free.

### High-Throughput Preparation of ASO-Loaded LNPs

LNP formulations were screened for different lipid compositions, total lipid concentrations, and ASO loading amounts that were designed in a 96-well plate matrix using the LEA Library Studio software (Unchained Labs, CA, USA). In a typical screening of ASO-1-loaded MC3 LNPs, the ASO was dissolved in citrate buffer (25 mM, pH 4) at concentrations corresponding to N/P ratios of 5, 2, 1, and 0.5, and dispensed into a 96-well plate (Greiner Bio One 655101, NC, USA) at 150 μl/well using a robotic liquid handler (TECAN® Freedom EVO, NC, USA). Lipid mixtures with varying total lipid amounts (0.2 or 0.4 μmol/well) and DSPE-PEG2000 contents (0, 1.5, 3, or 5 mol % of total lipids) were prepared by mixing individual lipid stocks (20 mg/ml in ethanol) and diluting with ethanol using the TECAN® robot. Then, 50 μl of lipids were rapidly dispensed into the ASO plate at 0.5 ml/s, followed by phase mixing by 10 rounds of pipetting (100 μl each time) using the TECAN® robot to promote self-assembly of ASO-loaded LNPs. The resulting plate contained 96 LNP samples (200 μl/well) varied with 32 conditions in parallel (4 levels of ASO loading, 2 levels of total lipid concentrations, and 4 levels of lipid compositions, n=3). In other experiments, the ionizable lipid MC3 was replaced by the permanently cationic lipid DOTAP, or the 13-mer ASO-1 was replaced by the 16-mer ASO-2, and screened for similar formulation parameters. The reverse dispensing sequence (injection of ASO solution into lipid mixtures) and different mixing speeds and rounds were also explored to optimize the phase mixing process.

### Characterization of ASO-Loaded LNPs

Structure of ASO-loaded LNPs was determined by a cryo-transmission electron microscope (cyro-TEM). DLS was used to measure particle size distributions. In brief, ASO-loaded LNPs were diluted ×40 in phosphate buffered saline (PBS, pH 7.4) in a 96-well, glass-bottom microplate (Greiner Bio One 655892, NC, USA) using the TECAN® robot, and analyzed for mean particle diameters and particle size distributions (presented by percent polydispersity, % PD) by using a DynaPro® plate reader III (Wyatt Technology, CA, USA). Sixty μl aliquots were adjusted to neutral pH by adding 15 μl of 0.5 M phosphate buffer (pH 7.4), then transferred to a filter plate (MWCO 100 kD; AcroPrep, PALL, NY, USA) and centrifuged (2,000×g, 10 min) for filtrates. The un-encapsulated ASO in 50 μl of filtrates was then quantified by OD260 using a UV plate reader (TECAN® Spark, NC, USA) and calculated for the percent encapsulation efficiency (% EE) of ASO:

$$\% EE = \frac{Total ASO amount - free ASO amount}{Total ASO amount} \times 100 \%$$

ASO standards were prepared in the same buffer and subjected to the same filtration process as LNP samples. For stability experiments, 60 μl of LNPs prepared under the N/P ratio of 1 was directly diluted ×10 in PBS and stored under 4 or 40° C., and analyzed for particle sizes and ASO release over 2 weeks.

### Microfluidic Preparation of ASO-Loaded LNPs

Microfluidic approach was used for scale-up preparation of ASO-loaded LNPs screened by the high-throughput approach described above. In brief, different concentrations of ASO-1 (dissolved in citrate buffer) and lipids (dissolved in ethanol) varying with total lipid concentrations and DSPE-PEG₂₀₀₀ contents were mixed by a microfluidic device (NanoAssemblr®, Precision NanoSystems, BC, Canada) under an aqueous buffer/ethanol phase ratio of 3/1 and a constant total flow rate of 12 ml/min. The collected LNPs were purified by centrifuge-based (2,000×g, 30 min) ultrafiltration (MWCO 10 kD; Amicon, MilliporeSigma, MA, USA) to remove free ASOs and lipids followed by buffer exchange to PBS. LNPs were analyzed for particle size distributions by DLS and ASO encapsulation by hydrophilic interaction liquid chromatography (HILIC). In brief, encapsulated ASOs were extracted from purified LNPs by dissolving in 0.75% Triton solution. A HILIC column (Waters ACQUITY UPLC BEH Amide, 130 Å, 1.7 μm, 3 mm×50 mm), mobile phase A (25 mM ammonium acetate in acetonitrile/water of 80/20, v/v), and mobile phase B (25 mM ammonium acetate in acetonitrile/water of 40/60, v/v) were used for gradient elution from 0-100% of phase B within 10 min, under a flow rate of 0.8 ml/min, column temperature of 40° C., and detection wavelength of 260 nm.

### Statistical Analysis

All results are presented as mean±SD, n=3. Data were analyzed by one-way or two-way analysis of variance (ANOVA) followed by Turkey's, Sidak's, or Dunnett's post tests for comparison of multiple groups using the Prism 8.0 (GraphPad Software). P values less than 0.05 were considered statistically significant.

### Example 1

### Optimization of the Phase Mixing Process by a Robotic Liquid Handler

To develop a high-throughput solvent-injection method for LNP preparation, the effects of phase mixing on particle size and ASO encapsulation were first investigated. ASO-1 was loaded in LNPs composed of 0.4 μmol of total lipids and 1.5 mol % of DSPE-PEG₂₀₀₀ through charge-mediated complexation under a N/P ratio of 1. The ethanol phase containing lipids was dispensed and mixed with the aqueous ASO phase, or vice versa, using a TECAN® liquid handler at different pipetting speeds ranging from the minimal 0.1 ml/s to the maximal 0.9 ml/s, according to the instrument settings. The ethanol-to-buffer injection produced similar LNPs with a mean diameter ˜145 nm (FIG. 1A), % PD˜18% (FIG. 1), and % EE of ASO˜83% (FIG. 1C) under low, medium, or high speed for the injection followed by 10 rounds of mixing. In contrast, buffer-to-ethanol injection at the low speed (0.1 ml/s) produced larger (mean diameter ˜220 nm) and more polydispersed (% PD˜41%) particles with lower % EE (˜43%) (FIGS. 1A-1C). However, increasing the injection speed produced similar LNPs as those from ethanol-to-buffer injections, suggesting fast dissipation of concentrated lipids in an aqueous buffer was required for the formation of ASO-loaded LNPs. Next, LNPs were prepared under the ethanol-to-buffer injection followed by phase mixing under different pipetting rounds and speeds. The medium speed (0.5 ml/s) and 10 rounds of mixing were sufficient to produce homogeneous LNPs with high ASO loading, whereas further increases in the mixing speed or rounds did not affect particle size and % EE (FIGS. 1D-1F). Therefore, the condition of ethanol-to-buffer injection followed by 10 rounds of mixing at 0.5 ml/s was chosen for following studies.

### Example 2

### HTS of ASO-Loaded LNP Formulations

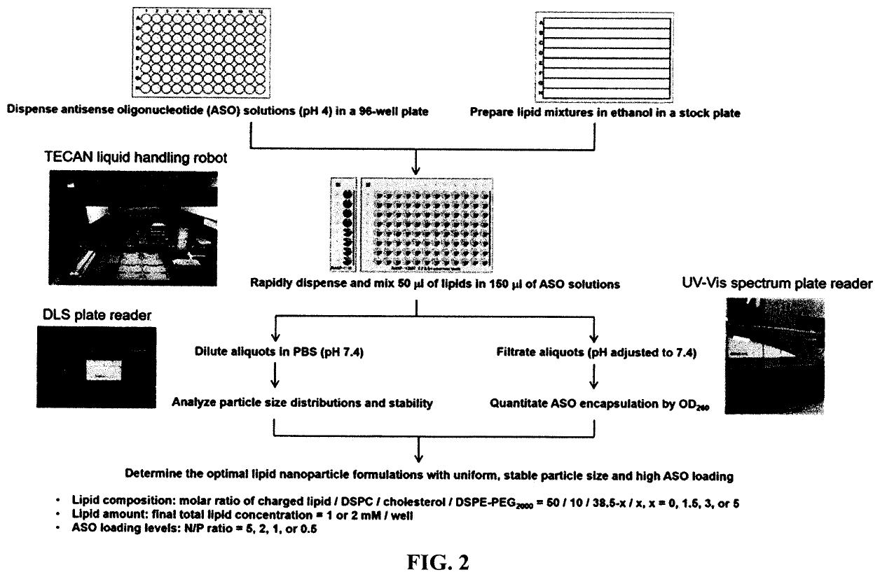

To investigate the impacts of formulation parameters on primary quality attributes of LNPs, a HTS workflow allowing streamline preparation and characterization of these formulations was designed (FIG. 2). ASO was initially dissolved in the citrate buffer with pH 4.0, which is below the pKa (6.4) of MC3 so that the lipid would bear positive charges to promote charge-mediated complexation. The solution pH was then adjusted to neutral by phosphate buffers before following analyses.

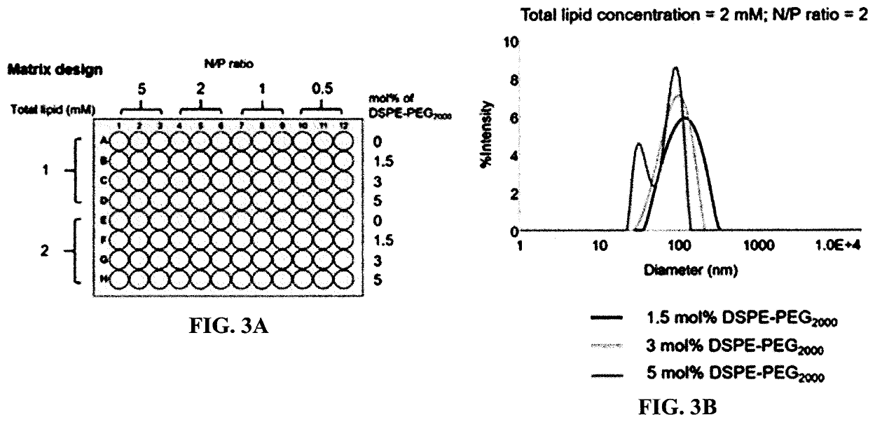

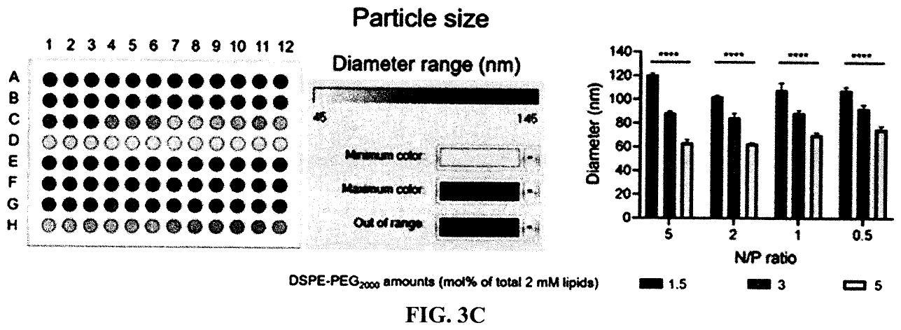

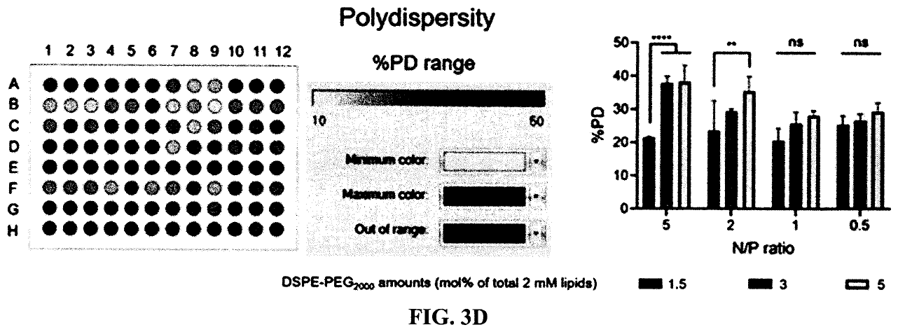

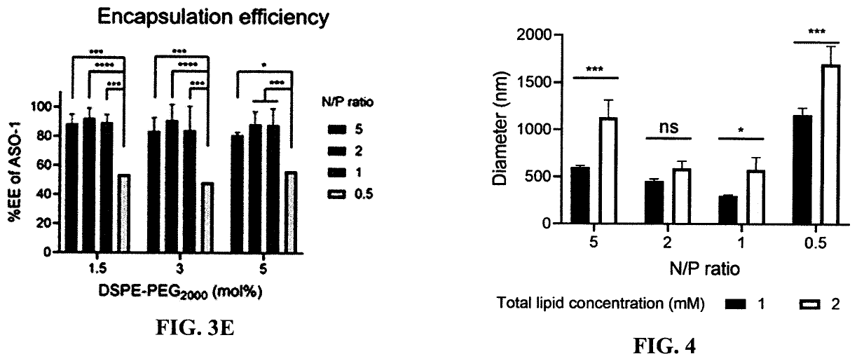

For a typical screening, 32 different samples (3 replicates each) varying with 2 levels of total lipid concentrations, 4 levels of ASO loading controlled by N/P ratios, and 4 levels of the PEGylated lipid content were screened in parallel in a 96-well plate (FIG. 3A). Among the three formulation parameters investigated, the PEGylated lipid was indispensable for LNP formation since there were multimodal, large aggregates produced when there was no PEG incorporated in the lipid composition (FIGS. 3C-3D, and FIG. 4). Increasing the PEGylated lipid content significantly (P<0.0001) reduced the mean particle size, i.e. lipids containing 1.5, 3, and 5 mol % of DSPE-PEG₂₀₀₀ resulted in LNP diameters of ˜120, ˜80, and ˜60 nm, respectively (FIGS. 3C-3D). However, polydispersity also increased, and 5 mol % of DSPE-PEG₂₀₀₀ even produced a subpopulation, possibly due to the formation of small DSPE-PEG₂₀₀₀ micelles (FIG. 3C). See, e.g., Johnsson et al., 2003, Biophys J 85(6):3839-47; Gill et al., 2015, J Drug Target 23(3):222-31.

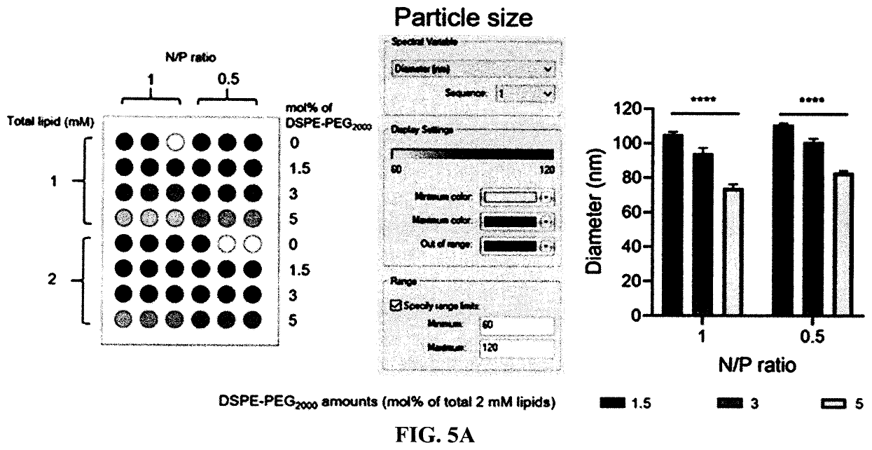

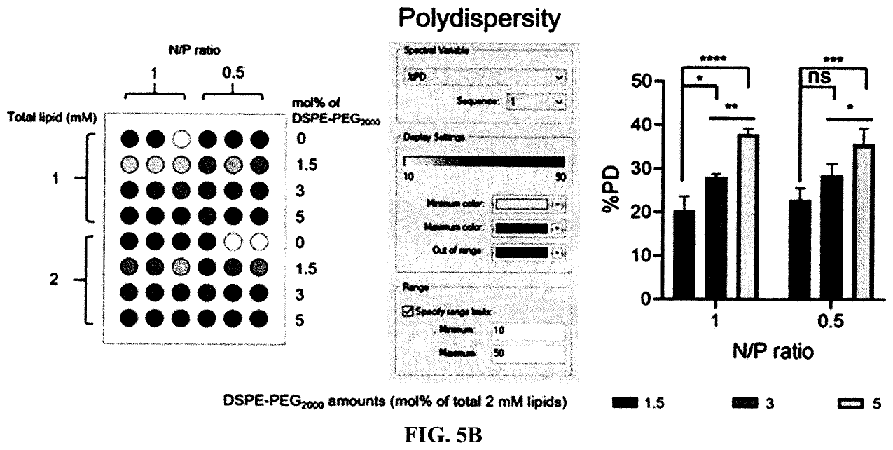

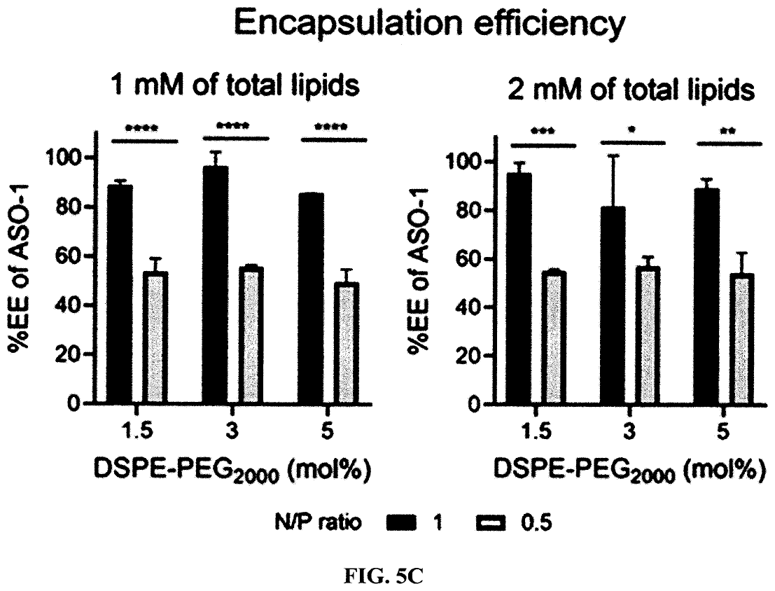

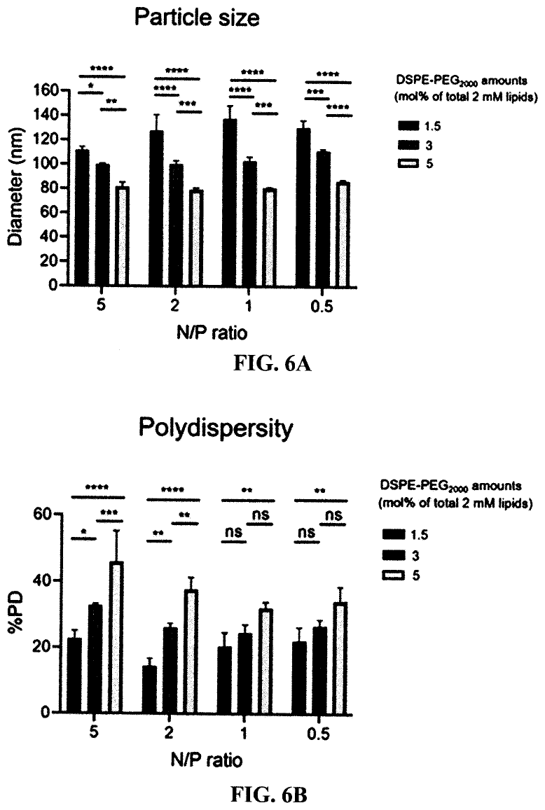

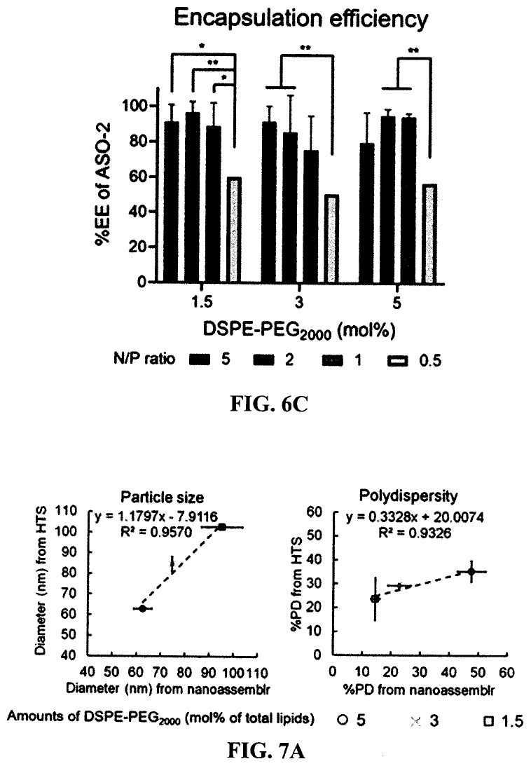

On the other hand, % EE of ASO was mainly determined by the N/P ratios. A N/P ratio higher than 1 with excess complexation sites in MC3 resulted in % EE>80%; whereas two-fold excess amounts of ASO-1 above the charge balance point significantly reduced % EE to ˜50% (FIG. 3E). Similar results were also found when MC3 was replaced by another cationic lipid DOTAP (FIGS. 5A-5C) or ASO-1 was replaced by ASO-2 (FIGS. 6A-6C), demonstrating the robustness of HTS results.

### Example 3

Validation of HTS Results with Scale-Up LNP Preparation

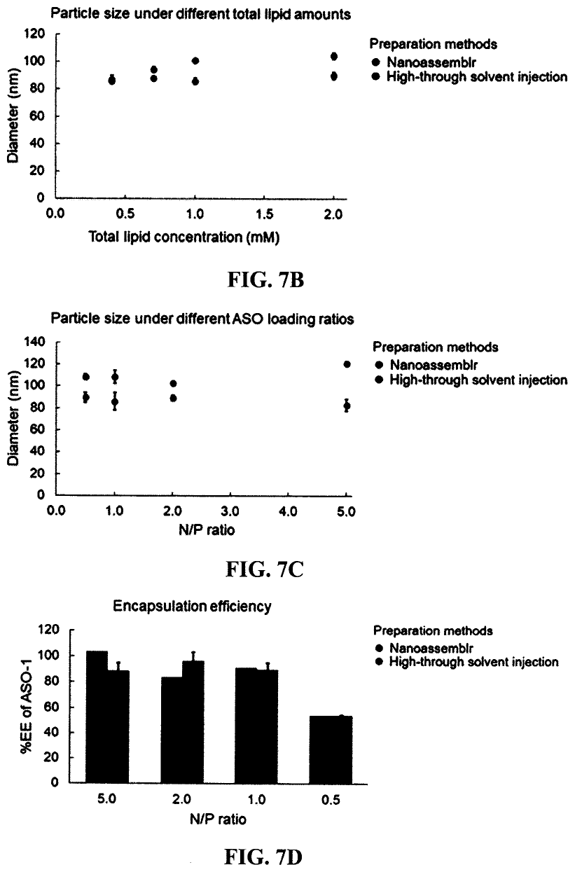

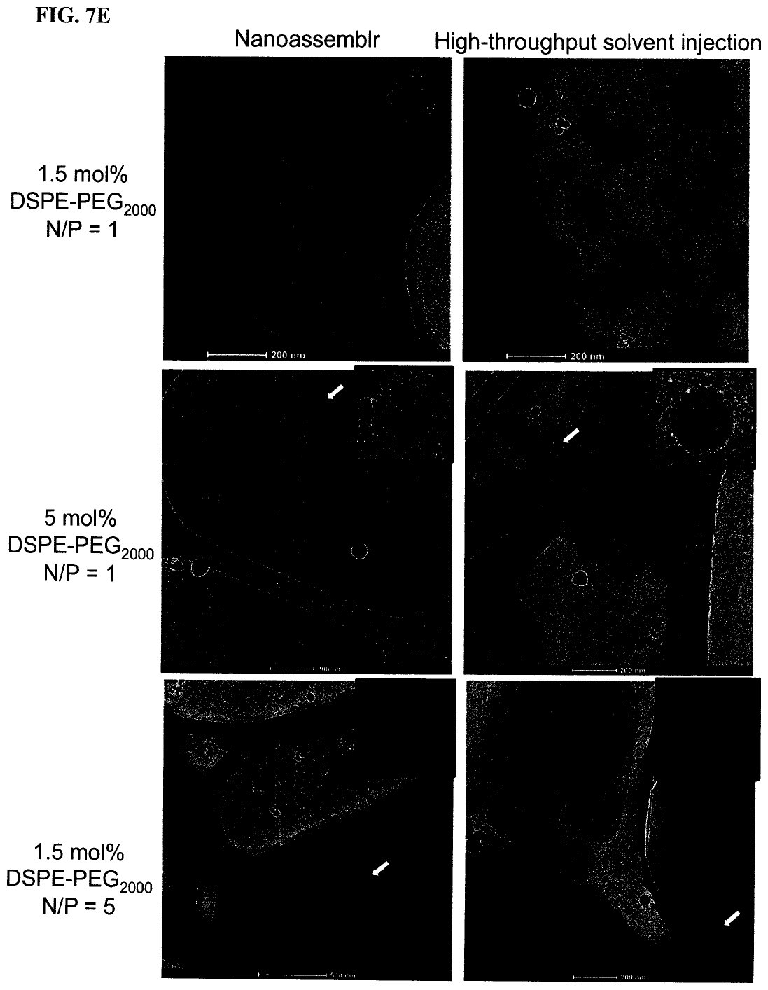

The influence of screened formulation parameters on LNP quality attributes was then validated by comparing results from the HTS approach with those from a microfluidic formulator. The two methods showed similar results: (1) LNP size decreased but polydispersity increased with increasing PEG contents (FIG. 7A); (2) LNP size were stable with increasing total lipid concentrations upto 2 mM (FIG. 7B); (3) LNP size remained stable when N/P ratio<2 (FIG. 7C); (4) excess ASO loading (N/P ratio<1) resulted in significant decrease in % EE (FIG. 7D); and (5) LNPs showed similar structures prepared with the same N/P ratio and PEGylated lipid content (FIG. 7E). Further, the HTS approach successfully predicted the dependence of particle size and polydispersity on the PEGylate lipid content, shown by strong correlations with linear regression R²>0.9 (FIG. 7A).

### Example 4

### Stability Screening of ASO-Loaded LNPs

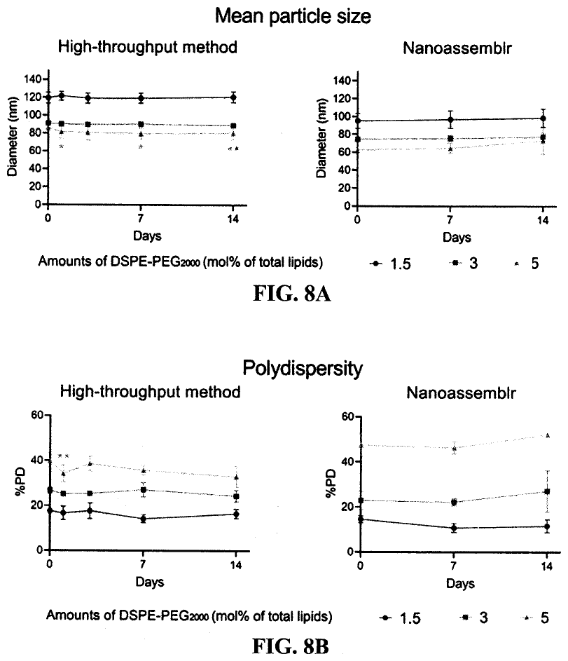

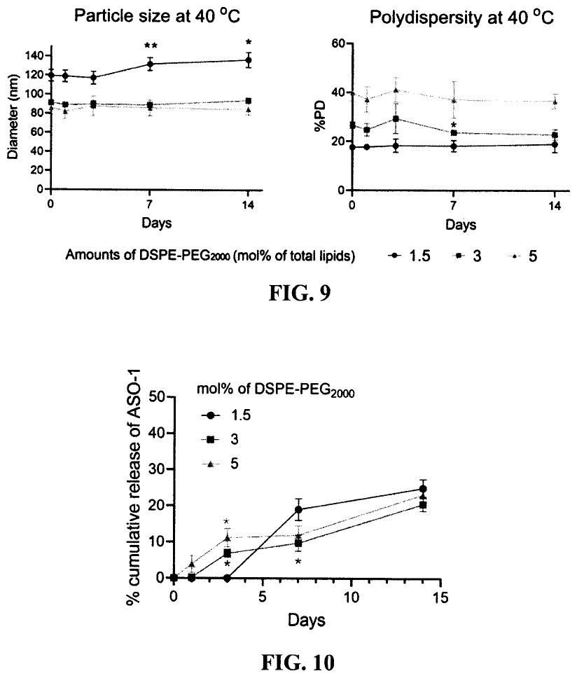

To further investigate the influence of different particle sizes on the formulation stability, ASO-1-loaded LNPs prepared with varying PEG contents were diluted by 10 times in PBS, incubated at 4° C. or 40° C., and particle size distributions over 2 weeks were monitored. The N/P ratio was kept ≥1 and % EE of ASO was ˜90%, so that ASO leakage from LNPs during the stability study could be quantified. As shown in FIGS. 8A-8B, LNPs prepared by high-throughput solvent injection or NanoAssemblr® with 1.5 or 3 mol % of DSPE-PEG₂₀₀₀ similarly remained their initial mean particle sizes (FIG. 8A) and polydispersity (FIG. 8B) during incubation at 4° C. At 40° C., LNPs containing 1.5 mol % DSPE-PEG₂₀₀₀ showed a particle size increase after 1 week, while remained constant polydispersity (FIG. 9). LNPs with 1.5 mol % of DSPE-PEG₂₀₀₀ also showed minimal ASO leakage within the first 3 days but similar levels of ASO leakage as LNPs with 3 and 5 mol % of DSPE-PEG₂₀₀₀ at 2 weeks after (FIG. 10). ASO leakage at 4° C. was not detected over 1 month.

The solvent-injection method for high-throughput preparation of LNP formulations was chosen since the phase mixing process could be executed by a robotic liquid handler. Compared with manual pipetting, a multichannel liquid handler allowed high-throughput, parallel processing of 96 samples and achieved uniform liquid dispense and mixing across wells. The key process involved fast and thorough mixing of intermiscible phases, e.g. ethanol dissolving lipids and an aqueous buffer dissolving the nucleic acids, in order to promote self-assembly of lipids into spherical lipid layers and nanoparticle structures. This method has been widely used to prepare liposomes, generating homogeneous nanoparticles when the ethanol phase was controlled under 50 vol %. Increasing the ethanol phase ratio and/or lipid concentrations produced large particles or aggregates probably due to inefficient phase mixing, as also shown by results of the low-speed, buffer-to-ethanol injection (FIGS. 1A-1B). The findings from the automated mixing process by the liquid handler were highly correlated with the results of LNPs prepared by the microfluidic method. The flow rate ratio (FRR, aqueous-to-organic flow rate) is one of the critical formulation parameters during the microfluidic preparation and a low FRR produces larger particles. The buffer-to-ethanol injection at the low speed represented the condition of low FRR. Therefore, the automated mixing conditions were optimized and the ethanol-to-buffer injection was set at 0.5 ml/s, under an ethanol/aqueous volume ratio of 1/3 (25 vol % of ethanol), followed by 10 rounds of pipetting to achieve efficient phase mixing and generate homogeneous particles with a high encapsulation efficiency.

Next, a streamline workflow was developed to screen formulation variables, including the total lipid concentration, lipid composition, and ASO loading amount, for optimal quality attributes of ASO-loaded LNPs. To this end, particle size distribution and % EE of ASO were measured by high-throughput DLS and OD260, respectively, to determine a condition that could produce homogeneous nanoparticles with high ASO loading. The screening results indicated that the PEGylated lipid content significantly affected particle size distributions (FIGS. 3B-3D, 5A-5B, and 6A-6B). DSPE-PEG₂₀₀₀ incorporated at 1.5 mol % of the total lipids produced unimodal nanoparticles with a mean diameter of ˜120 nm, whereas more PEG increased polydispersity. Ionizable lipids consisting of tertiary amine structures have been increasingly used for lipid-based delivery systems for nucleotides, showing better intracellular delivery efficiency and lower cytotoxicity than permanently charged cationic lipids. See, e.g., Cullis & Hope, 2017, Mol. Ther. 25(7):1467-1475, Sabnis et al. 2018, Mol Ther. 26(6):1509-1519; Semple et al., 2010, Nature Biotechnology, 28(2): 172-176. In line with the loading mechanism of charge-mediated complexation, the screening results indicated N/P ratios determined ASO encapsulation, showing % EE of ˜90% at N/P ratio=1 (FIGS. 3E, 5C, and 6C), corresponding to a loading capacity of 0.29 mg RTR3833/mg lipids (2 mM total lipids with 1.5 mol % of DSPE-PEG₂₀₀₀).

Importantly, results from the HTS approach successfully predicted those from a microfluidic formulator, which has been increasingly utilized to prepare nanoparticle formulations with scalable productions. See, e.g., Belliveau et al., 2012, Mol. Ther. Nucleic Acids, 1, e37; van Swaay & deMellow, 2013, Lab Chip 13(5):752-67. Both methods showed similar dependence of LNP size on PEGylated lipid contents (FIG. 7A), total lipid concentration (FIG. 7B), and N/P ratios (FIG. 7C), as well as the % EE of ASO were similarly controlled by N/P ratios (FIG. 7D). The two methods also produced LNPs with similar structures under the same formulation parameters (FIG. 7E). Further, these ASO-loaded LNPs showed stable particle size distributions (FIGS. 8A-8B) and ˜20% leakage of the encapsulated ASO over 2-week storage at 40° C. (FIG. 10). Compared with microfluidic preparation, however, the HTS approach showed significant advantages in saving raw materials by ˜10 fold, while increasing preparation and analytical outputs by ˜100 fold (parallel processing 96 samples in microplates compared with single microfluidic run), indicating its great potential for early-stage formulation screenings (FIG. 11). Based on the screening results, it was determined that 1.5 mol % of DSPE-PEG₂₀₀₀ and the N/P ratio≥1 would produce optimal LNP formulations with a homogeneous and stable particle size as well as high ASO loading. The same statement was still valid after introducing different lipids and other ASOs into the HTS system, which suggested that this screening platform could expand their applications to various types of carriers and cargos, such as siRNAs and single-guided RNA.

The HTS screening approach demonstrated a reproducible formulation platform to prepare LNPs. The translatable outcomes from the automated injection platform to microfluidic preparations created a seamless workflow to support screening and scale-up formulations, and avoided bridging studies arising from formulation inconsistency. The next step is to integrate the current workflow with downstream in vitro screenings to correlate physicochemical attributes of ASO-loaded LNPs with their therapeutic efficacy. In addition, the workflow could be further improved to address more formulation attributes, such as zeta potential and simultaneous quantification of both API and excipients by liquid chromatography strategies. Yamamoto et al., 2011 J Chromatogr B Analyt Technol Biomed Life Sci 879(20), 3620-5, Li et al., 2019, J Chromator A 1601:145-154.

In this example, a high-throughput approach to screen formulation parameters and address quality attributes of ASO-loaded LNPs was developed. The streamline workflow starting from automated liquid dispense and mixing, followed by high-throughput particle size and ASO encapsulation analyses identified the PEGylated lipid content and N/P ratio were the primary determinants of particle size distribution and encapsulation efficiency, respectively. Furthermore, the HTS results successfully predicated those from scale-up preparation using microfluidics. The robust screening results, as well as significant material saving and improvement in analytical outputs suggest great promise of this approach to advance the development of lipid-based nanoparticle formulations.

### Example 5

### Alternative Method of Quantification of ASO Encapsulation

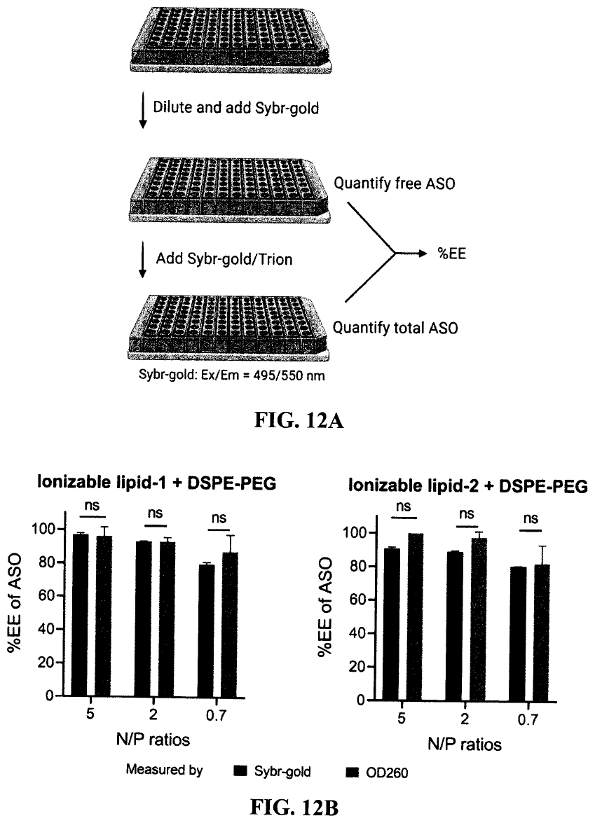

The quantification of ASO encapsulation was determined using a fluorescence plate reader. In brief, ASO-loaded LNPs were prepared by the high-throughput solvent injection method, and then diluted 50× in TE buffer, mixed with an equal volume of 5000× diluted fluorescence probe Sybr-gold, and the unencapsulated ASO was quantified using a fluorescence plate reader (Ex/Em=495/550 nm). The LNPs were then disrupted by the direct addition of an equal volume of 10000× diluted Sybr-gold in 1 vol % Triton TE (i.e., the final probe dilution was kept at 10000× and Triton concentration was 0.5 vol %) (FIG. 12A). The fluorescence measurement was then taken to quantify the total ASO. Percent encapsulation efficiency (% EE) was calculated as:

$$\% EE = \frac{Total ASO amount - free ASO amount}{Total ASO amount} \times 100 \%$$

Calculations showed comparable % EE results for two different LNP formulations prepared under different N/P ratios using the fluorescence and UV-Vis methods (FIG. 12B). Results are presented as mean±SD, n=2; ns, not significant, analyzed by two-way ANOVA followed by Sidak's multiple comparison.

### Example 6

### HTS of HiBiT Peptide-Loaded LNP Formulations

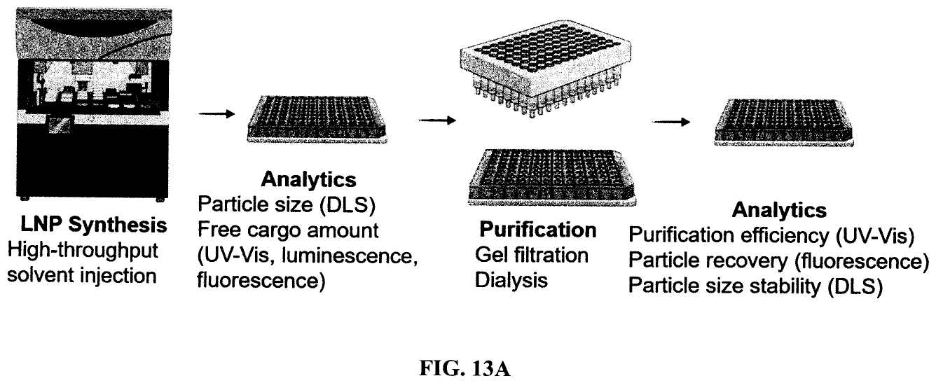

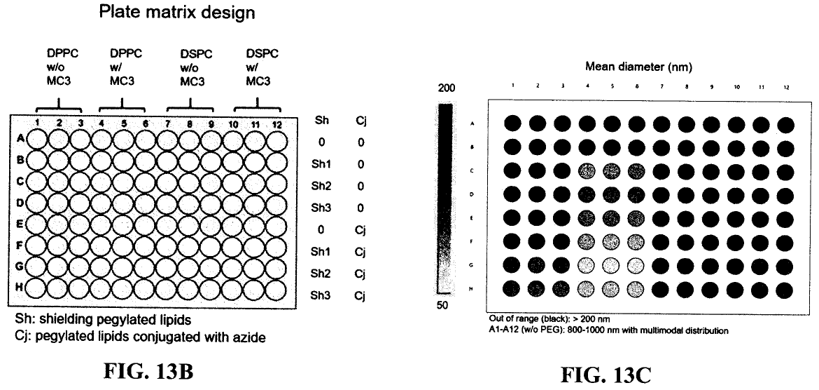

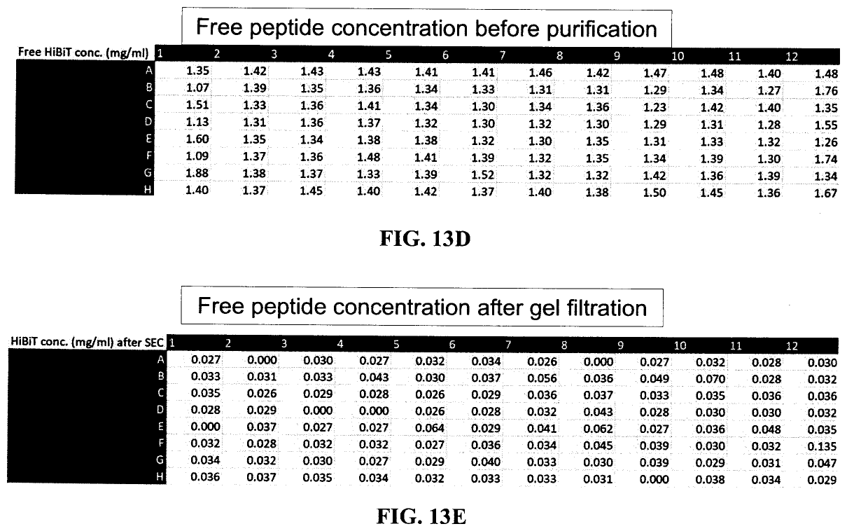

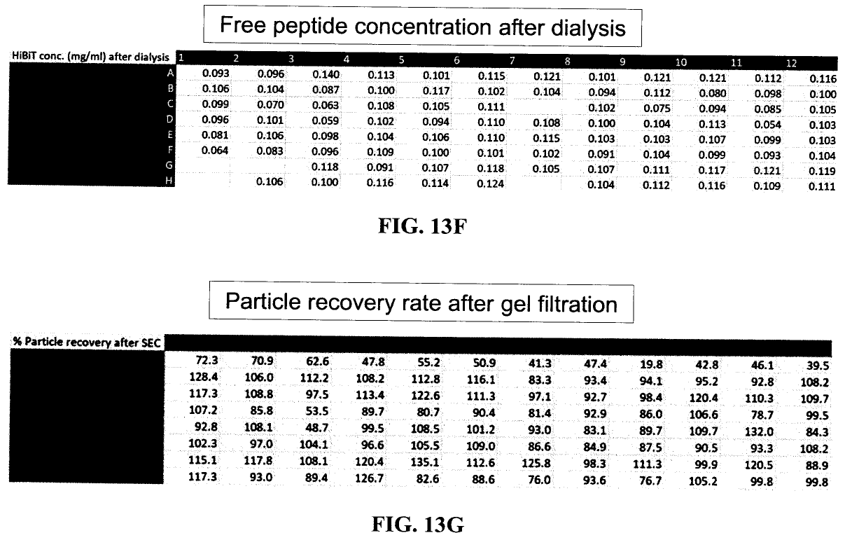

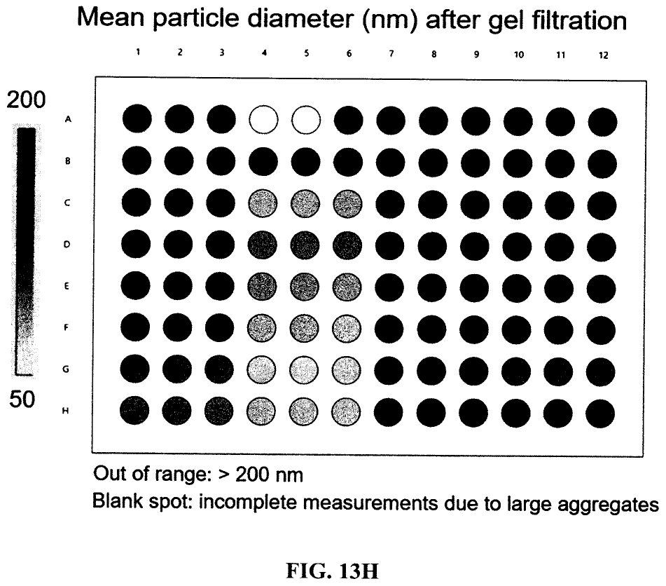

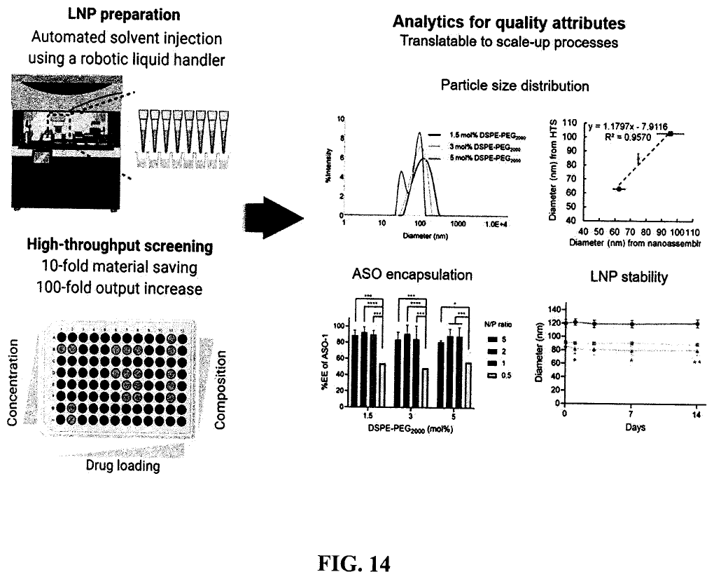

To investigate the impacts of formulation parameters on primary quality attributes of liposomes, an HTS workflow allowing for streamlined preparation and characterization of these formulations was designed. HiBiT was initially dissolved in the 20 mM histidine-acetate buffer supplemented with 150 mM NaCl (pH 5.5) and dispensed into microwell plates using a robotic liquid handler. Lipid mixtures were prepared similarly as in the Example 2. (FIG. 13A).

For a typical screening, 32 different samples (3 replicates each) varying with 4 types of LNP formulations and 8 combinations of the PEGylated lipids, shielding pegylated lipids and pegylated lipids conjugated with azide, were screened in parallel in a 96-well plate (FIG. 13B). Among the 8 formulation parameters investigated, the PEGylated lipid was necessary for LNP formation since there were multimodal, large aggregates produced when there was no PEG incorporated in the lipid composition (FIG. 13C). The free peptide concentration quantifications before and after purification yielded mean purification efficiencies of ˜98% and ˜61% for gel filtration and dialysis, respectively (FIGS. 13D-13F). Particle recovery rates were generally between 80-120%, except for low values due to aggregated samples that were prepared without the pegylated lipids (FIG. 13G). Additionally, particle size distributions remained constant after purification by gel filtration (FIG. 13H).

### Example 7

HTS identified the impacts of diverse PEG-lipids attributes on ASO-LNP size distributions.

### HTS Preparation and Characterization of ASO-Loaded LNPs

Cholesterol, 1,2-distearoyl-sn-glycero-3-phosphocholine (DSPC), and all linear PEGylated lipids for LNP formulations were purchased from Avanti Polar Lipids (AL, USA). Branched PEGylated lipid N-[2′,3′-Bis(methylpolyoxyethyleneoxy)propane-1′-oxycarbonyl]-1,2-distearoyl-sn-glycero-3-phosphoethanolamine (DSPE-2arm-PEG-2k) was commercially sourced from NOF America Corporation (NY, USA). The complete list of PEGylated lipids is listed in Table 1 below.

TABLE 1
PEGylated lipid analogs used for the high-throughput screening of ASO-LNPs

| | | | | | | | PEG | |
|---|---|---|---|---|---|---|---|---|
| PEG | PEG-lipid | MW | | | C-tail | C-tail | Size | PEG |
| lipid # | name | (Da) | Lipid type | Charge | length | saturation | (Da) | Architecture |
| 1 | DMPE(C14:0)- | 1240 | Phosphoglyceride-PE | Negative | 14 | Saturated | 550 | Linear |
| | PEG_{0.55k} | | | | | | | |
| 2 | DMPE(C14:0)- | 1680 | Phosphoglyceride-PE | Negative | 14 | Saturated | 1000 | Linear |
| | PEG₁ₖ | | | | | | | |
| 3 | DMPE(C14:0)- | 2693 | Phosphoglyceride-PE | Negative | 14 | Saturated | 2000 | Linear |
| | PEG₂ₖ | | | | | | | |
| 4 | DPPE(C16:0)- | 1736 | Phosphoglyceride-PE | Negative | 16 | Saturated | 1000 | Linear |
| | PEG₁ₖ | | | | | | | |
| 5 | DPPE(C16:0)- | 2749 | Phosphoglyceride-PE | Negative | 16 | Saturated | 2000 | Linear |
| | PEG₂ₖ | | | | | | | |
| 6 | DSPE(C18:0)- | 1352 | Phosphoglyceride-PE | Negative | 18 | Saturated | 550 | Linear |
| | PEG_{0.55k} | | | | | | | |
| 7 | DSPE(C18:0)- | 1792 | Phosphoglyceride-PE | Negative | 18 | Saturated | 1000 | Linear |
| | PEG₁ₖ | | | | | | | |
| 8 | DSPE(C18:0)- | 2805 | Phosphoglyceride-PE | Negative | 18 | Saturated | 2000 | Linear |
| | PEG₂ₖ | | | | | | | |
| 9 | DSPE(C18:0)-2arm- | 2843 | Phosphoglyceride-PE | Negative | 18 | Saturated | 2000-B | Branched |
| | PEG₂ₖ | | | | | | | |
| 10 | DOPE(C18:1)- | 1348 | Phosphoglyceride-PE | Negative | 18 | Unsaturated | 550 | Linear |
| | PEG_{0.55k} | | | | | | | |
| 11 | DOPE(C18:1)- | 1788 | Phosphoglyceride-PE | Negative | 18 | Unsaturated | 1000 | Linear |
| | PEG₁ₖ | | | | | | | |
| 12 | DOPE(C18:1)- | 2801 | Phosphoglyceride-PE | Negative | 18 | Unsaturated | 2000 | Linear |
| | PEG₂ₖ | | | | | | | |
| 13 | DMG(C14:0)- | 2509 | Diglyceride | Neutral | 14 | Saturated | 2000 | Linear |
| | PEG₂ₖ | | | | | | | |
| 14 | DSG(C18:0)- | 2621 | Diglyceride | Neutral | 14 | Saturated | 2000 | Linear |
| | PEG₂ₖ | | | | | | | |
| 15 | Ceramide(C8)- | 1245 | Ceramide | Neutral | 8 | Saturated | 750 | Linear |
| | PEG_{0.75k} | | | | | | | |
| 16 | Ceramide(C8)- | 2522 | Ceramide | Neutral | 8 | Saturated | 2000 | Linear |
| | PEG₂ₖ | | | | | | | |
| 17 | Ceramide(C16)- | 1357 | Ceramide | Neutral | 16 | Saturated | 750 | Linear |
| | PEG_{0.75k} | | | | | | | |
| 18 | Ceramide(C16)- | 2634 | Ceramide | Neutral | 16 | Saturated | 2000 | Linear |
| | PEG₂ₖ | | | | | | | |

Ionizable lipid dilinoleylmethyl-4-dimethylaminobutyrate (DLin-MC3-DMA; MC3) for LNP formulation was purchased from MedChemExpress (NJ, USA). A 17-mer ASO (MW 5635 Da, Na-salt form) with phosphorothioate backbone was custom synthesized by BioSpring GmbH (Frankfurt, Germany) using solid-phase synthesis. All other reagents were DNase/RNase-free and used from their commercial sources without further purification.

LNPs with various PEGylated lipids were prepared using a high-throughput approach reported previously. Briefly, the ASO was dissolved at 93.9 μg/mL in citrate buffer (25 mM, pH 4.0) and dispensed at 150 μL/well in a 96-well plate (Greiner Bio One 655101, NC, USA) using a TECAN Freedom EVO® robotic liquid handler (Tecan Life Sciences, NC, USA). Using the automated setup, different lipid mixtures composed of MC3, DSPC, cholesterol, and respective PEG-lipid analogs were prepared in ethanol at a molar ratio of 40:10:(50−X):X, where X=1, 3, or 5; and a total lipid concentration of 4 mM to maintain N:P=2 for the resulting ASO-LNPs. N:P is defined as the molar ratio of positively-chargeable amine (N) groups in the ionizable lipid to negatively-charged phosphate (P) groups on the nucleic acid backbone. The lipid mixtures were transferred to a 12-channel reservoir plate (Axygen RES-MW12-LP or —HP, NC, USA), and 50 μL of the lipid phases were injected into the ASO solutions in the 96-well plate using the robot (speed=0.5 mL/s, followed by 10 cycles of mixing with 0.1 mL/cycle), resulting in 1 mM total lipids/well at ethanol: aqueous phase volume ratio of 1:3. Each LNP formulation was prepared in triplicate. The ASO-loaded LNPs were diluted in phosphate-buffered saline (PBS, pH 7.4) to achieve a final concentration of 1 mM total ASO/well. Small aliquots of each sample were transferred into a glass-bottom 96-well plate (Greiner Bio One 655892, NC, USA) and further diluted 50× in PBS for characterization of their particle size distributions by dynamic light scattering (DLS) using a DynaPro® plate reader III (Wyatt Technology, CA, USA).

### Microfluidic Preparation and Characterization of ASO-Loaded LNPs

A microfluidic mixing method was used for the scale-up preparation of selected positive and negative hit ASO-LNP formulations identified by the HTS approach. Different lipid mixtures composed of MC3, DSPC, cholesterol, and selective PEG-lipid analogs were dissolved in ethanol at a molar ratio of 40:10:(50−X):X, where X=1, 3, or 5, and a total lipid concentration of 4 mM. The ethanol stream was rapidly mixed with an aqueous stream containing 93.9 μg/mL ASO dissolved in citrate buffer (25 mM, pH 4.0) using a microfluidic laminar mixing device (NanoAssemblr™ Benchtop, Precision NanoSystems, BC, Canada) at a 1:3 volume ratio, and a total flow rate of 12 mL/min. The formulated LNPs were purified by centrifugal ultrafiltration (MWCO 10 kDa; Amicon, MilliporeSigma, MA, USA) at 2,000 g for 30 min to remove free ASO and lipids, followed by buffer exchange to RNase-free PBS. Purified formulations were analyzed for their particle size distribution using DLS.

### Statistical Analysis

Data plotting and statistical analysis were performed using Prism 9.2.0 (GraphPad Software, San Diego, CA). All results are presented as mean±SEM, n=3 containing 3 averaged internal replicates.

### Diverse PEG-Lipids Used for ASO-LNP Formulation Library

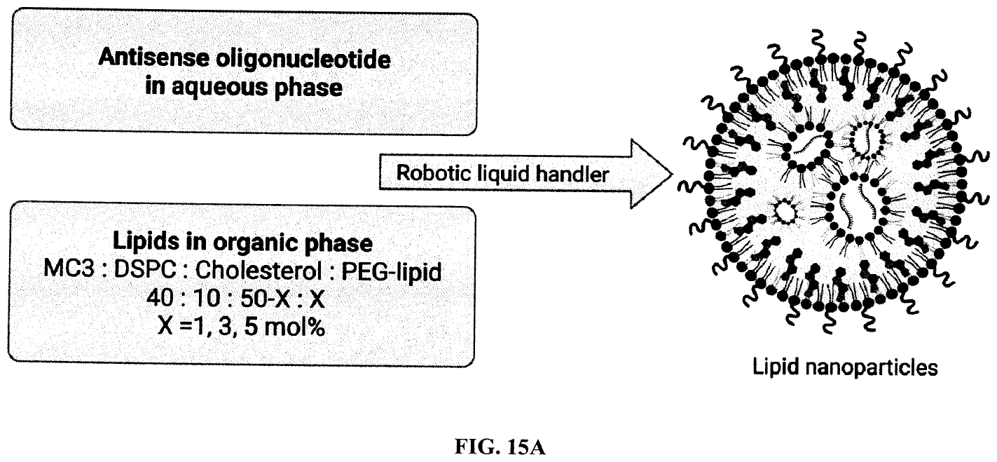

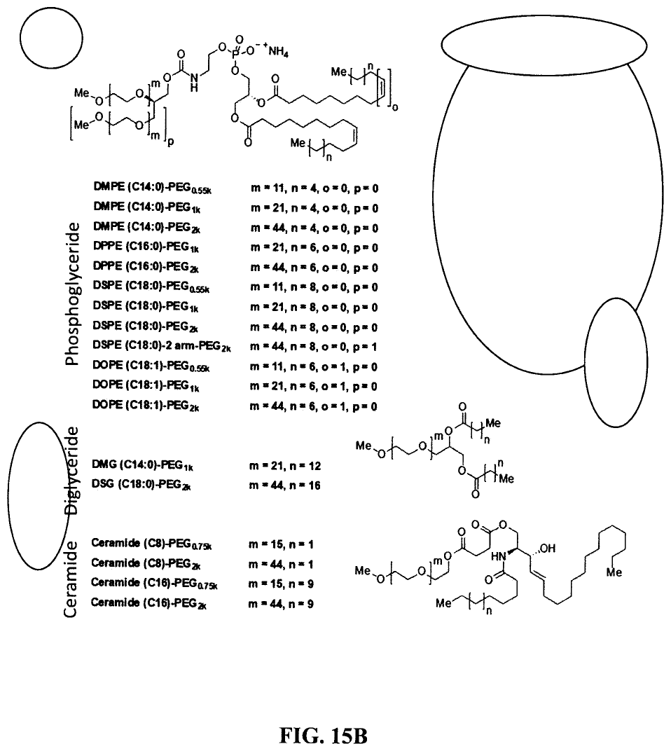

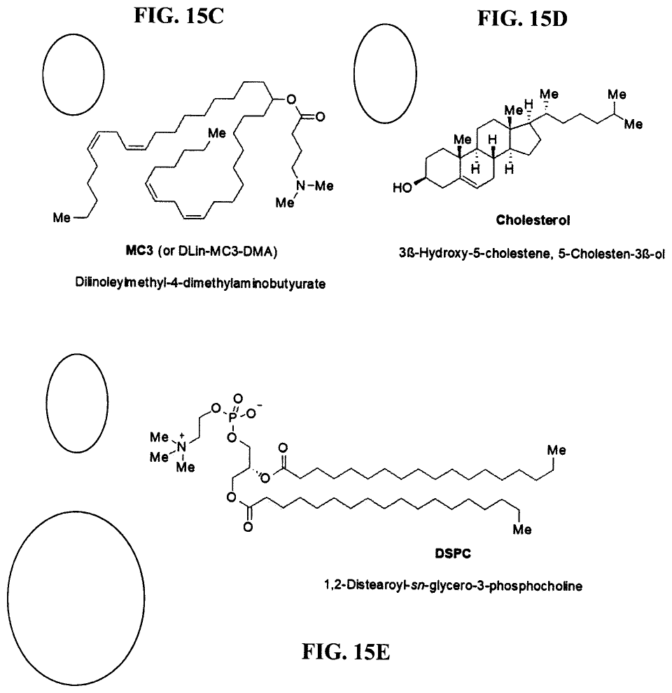

In the present HTS lipid library design, we chose DLin-MC3-DMA (MC3), DSPC, and cholesterol as the constituent lipids for all ASO-LNP formulations, while the PEG-lipid content was varied (FIGS. 15A and 15B). The combination of MC3, DSPC, and cholesterol is used in ONPATTRO®, the first FDA-approved siRNA-LNP formulation, and is used as an oligonucleotide delivery benchmark in many preclinical LNP model systems. To systematically understand the effect of PEG-lipid properties on LNP cargo delivery, several PEG-lipid analogs were used in combination with MC3, DSPC, and cholesterol during ASO-LNP formation (Table 1). PEG-lipids commonly used in drug delivery applications were chosen from biologically relevant lipid families including the anionic phosphoglycerides, and neutrally charged diglycerides and ceramides. We included multiple analogs for each PEG-lipid type, with varying C-tail lengths or PEG chain sizes (FIG. 15B). The impact of architecture (linear or branched) and PEG-lipid C-tail saturation were also assessed in this study.

In addition to testing individual PEG-lipid features, we also varied PEG-lipid concentration to assess its impact on formulation characteristics and cargo delivery. The mole ratio of PEG-lipid in the LNPs was tuned to 1, 3, and 5 mol % by adjusting the mole ratio of cholesterol in the lipid mixture. Using these PEG-lipid parameters, a library of 54 distinct ASO-LNP formulations was prepared in a 96-well plate-based HTS workflow for physicochemical characterization and in vitro evaluation of ASO delivery.

### PEG Content Governs the Particle Size Distribution of ASO-LNPs

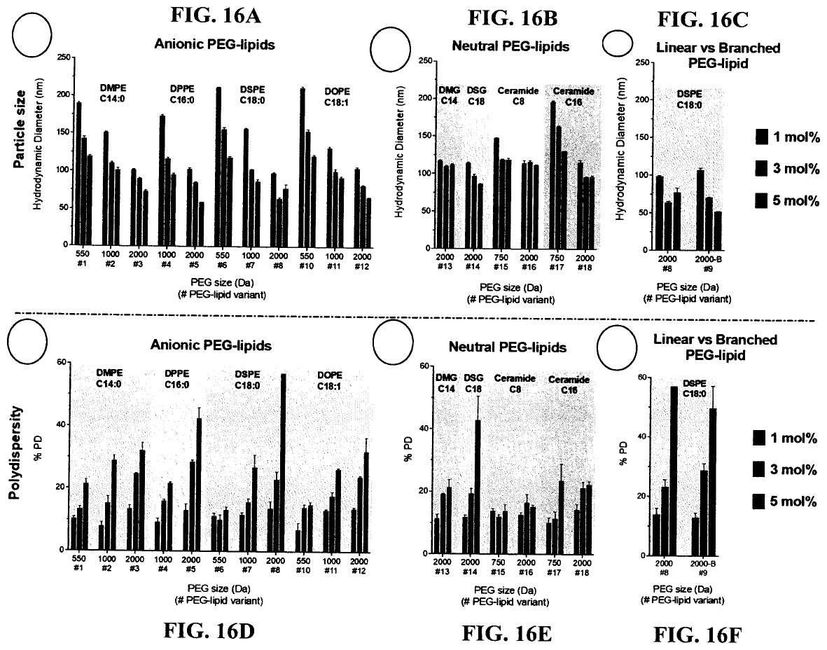

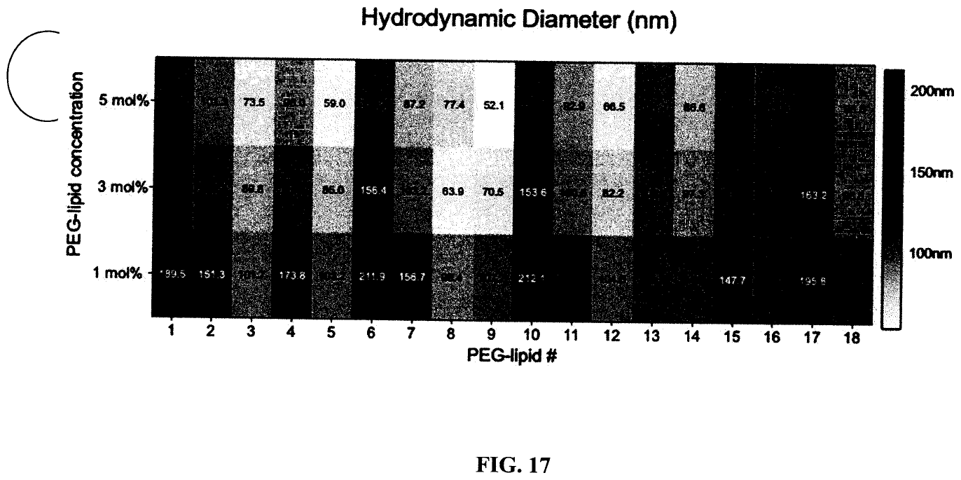

The mean hydrodynamic diameters of 54 distinct ASO-LNP formulations ranged between 52 and 212 nm as measured by DLS, and showed a general trend of decreasing particle sizes with increasing molar ratios of the PEGylated lipids from 1% to 5% (FIGS. 16A-16C). LNPs with short PEG-lipids (M.W.<1000 Da) formulated at 1 mol %, e.g. #6 and #10, had the largest particle diameter at 212 nm. In contrast, 5 mol % of long PEG lipids (#9) produced the smallest particles (52 nm), likely due to an increased steric hindrance by the PEG chains to prevent particle growth. Specifically, anionic PEG lipids showed a distinct decrease in particle sizes, with increasing PEG sizes, as well as PEG molar ratios in the LNP composition (FIG. 16A). In contrast, LNPs prepared with neutral diglyceride and ceramide PEG-lipids did not show a correlation between particle size and PEG content, with particular non-correlation seen in formulation #13 and #16 (FIG. 16B). These findings suggest a major role of the repulsive forces between charged head groups in determining particle sizes of the anionic-PEGylated LNPs, alongside the steric barrier of the PEG chains. Additionally, a comparison of linear and branched DSPE-PEG₂k variants (#8 and #9) showed no significant effect of PEG architecture on hydrodynamic diameter (FIG. 16C). Mean particle diameters were also shown in a color-coded heatmap to indicate the size trends (See FIG. 17).

While increasing PEG-lipid molar ratio has charge-dependent effects on particle diameter, PEG-lipid content has an overall positive correlation with LNP polydispersity (% PD) (FIGS. 16D-16F). Notable exceptions to this trend were #6, #15, and #16. PEG-lipids #15 and #16 are from the neutrally charged ceramide-C8 PEG-lipid family, which also showed no correlation between PEG-lipid content and particle size. In agreement with our previous data, PEG-lipids formulated at 5 mol % with long PEG arms (2000 Da) had highly polydisperse size distributions, possibly due to presence of micellar PEG-lipid sub-populations.

We also compared the effect of lipidic anchor groups with different C-tail lengths (DMPE/DPPE/DSPE, DSG/DMG, and Cer-C8/Cer-C16), saturation levels (DSPE/DOPE), and PEG-linker chemistries (DMPE/DMG, and DSPE/DSG). PEG-lipid hydrophobic tails did not have a significant effect on LNP size or polydispersity indices, which contrasts with the importance of the hydrophilic PEG component of PEG-lipids. Similar observations have been previously reported for siRNA-LNPs prepared with PEG-lipids containing 14, 16, or 18-carbon chains.

### Regression Analysis on LNP Particle Sizes

Linear regression models corroborated the significance (p<0.05) of PEG-lipid charge, molar ratio, and PEG size on LNP particle size distributions, as compared to the non-significant impact of different C-tail attributes (Table 2 and Table 3).

### Linear Regression Model for Anionic PEG-Lipids:

$$ASO - LNP hydrodynamic diameter ( nm ) = 225.71 - {0.44}^{*} carbon tail length ( \# C ) - {0.05}^{*} PEG size ( Da ) - {14.12}^{*} PEG - lipid mol \%$$

TABLE 2

| | Coeffi- | Standard | | |
|---|---|---|---|---|
| | cients | Error | t Stat | P-value |
| Inter- | 225.7093718 | 26.01575913 | 8.67587106 | 6.4911E−10 |
| cept | | | | |
| C-tail | −0.445909286 | 1.509467593 | −0.2954083 | 0.76959034 |
| length | | | | |
| (#C) | | | | |
| PEG | −0.047938375 | 0.004190871 | −11.438761 | 7.6631E−13 |
| size | | | | |
| (Da) | | | | |
| PEG- | −14.12145396 | 1.568862698 | −9.001077 | 2.788E−10 |
| lipid | | | | |
| mol % | | | | |

### Model Summary:

R Square=0.869, Adjusted R Square=0.857, Standard Error=15.37, Observations=36. ANOVA: Significance F=3.16×10⁻¹⁴

### Linear Regression Model for Neutral PEG-Lipids:

$$ASO - LNP hydrodynamic diameter ( nm ) = 172.68 + 1.2 {9}^{*} carbon tail length ( \# C ) - 0. {3}^{*} PEG size ( Da ) - {6.21}^{*} PEG - lipid mol \%$$

TABLE 3

| | Coeffi- | Standard | | |
|---|---|---|---|---|
| | cients | Error | t Stat | P-value |
| Inter- | 172.6862673 | 17.81521134 | 9.693192182 | 1.3723E−07 |
| cept | | | | |
| C-tail | 1.294659428 | 1.101953097 | 1.174877072 | 0.25964028 |
| length | | | | |
| (#C) | | | | |
| PEG | −0.031757849 | 0.006357037 | −4.99569956 | 0.00019607 |
| size | | | | |
| (Da) | | | | |
| PEG- | −6.209444167 | 2.271734507 | −2.733349407 | 0.0161647 |
| lipid | | | | |
| mol % | | | | |

### Model Summary:

R Square=0.700, Adjusted R Square=0.636, Standard Error=15.74, Observations=18. ANOVA: Significance F=5.9×10⁻⁴

Taken together, these data suggest that LNP size distribution is primarily dependent on surface stabilizing PEG content rather than lipid-tail attributes, and this PEG-dependence is especially dominant in anionic PEG-lipid scaffolds.

### Example 8

### HTS for Identification of ASO-LNP Behavioral Trends

The HTS approach of the invention allows for quick preparation and characterization of diverse ASO-LNP formulations in a 96-well plate format. This high-throughput workflow can be seamlessly extended to evaluate LNP delivery in target cell lines. This HTS approach leads to significant material and time savings. Furthermore, it generates robust datasets by directly comparing ASO-LNP formulations in identical environments, thus minimizing processing variations.

The HTS approach also offers data analysis and interpretation advantages. First, characterization involving numerous formulations can be generated in short time frames (FIGS. 16A-16F). Second, comprehensive screening allows for identifying correlations that may be masked in narrow sample sizes. For instance, our HTS datasets indicate that hydrophilic PEG components of PEG-lipids govern the LNP particle size distributions (FIGS. 16A-16F). Such behavioral trends can be defined quantitatively through predictive correlations using regression analyses. Linear regression models substantiate the significant charge-dependent effect of the PEG-lipid concentrations and PEG sizes (p<0.05) on the LNP size distributions, compared to their C-tail attributes. The accuracy of these correlations can be further improved through iterative screening of wider sample sets, in combination with advanced machine learning algorithms.

### HTS Predicts ASO-LNP Performance of Scaled-Up Formulations

Like all screening assays, the value of the HTS approach resides in its ability to predict behavior in a scaled-up system. Therefore, we validated the translatability of LNP attributes from the HTS approach to a scalable microfluidics method to produce self-assembled nanoparticle formulations. Six hit ASO-LNPs formulated with specific PEG species and molar ratios (#13-1%, #16-3%, #1-5%, #10-1%, #8-3%, #9-5%) were chosen from the HTS library. The representative formulations were selected with a rationale to include a diverse PEG-lipid array comprising diglyceride (#13), ceramide (#16), and phosphoglyceride (#1) PEG-lipids with different lipid tail saturation levels (#10, #8), PEG architecture (#9), as well as PEG-lipid content (1, 3, 5 mol %), to ensure a robust validation of HTS predictability across a broad range of PEG content in LNPs. The selected ASO-LNPs were scaled up 10× using a microfluidic mixer (NanoAssemblr™ Benchtop) under similar formulation conditions as their HTS counterparts.

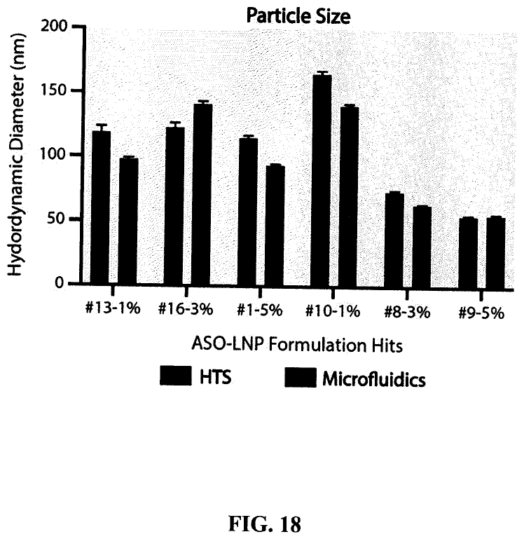

DLS characterization of the 6 different LNPs prepared with microfluidics showed that their hydrodynamic diameters were similar to the respective LNPs formulated using the HTS method. (FIG. 18). The HTS approach reliably predicted the physicochemical properties and provided a smooth translation of these attributes in microfluidics-based formulations. Currently, microfluidics technology has been adapted widely due to its scalability (from <mL to L) suitable for the preclinical and clinical translation of LNP-based drug delivery vehicles. Thus, the predictive HTS approach can be utilized to scale up formulations while saving significant resources typically required for optimizing formulations on scalable microfluidics platforms.

In accordance with the invention, a high-throughput screening approach is described to characterize LNP size distribution trends as a function of various PEG-lipid parameters like the PEG size, PEG-lipid content in LNP, carbon-tail length, etc. The invention described herein can further be combined with machine learning algorithms to identify and define quantitative correlations across the huge datasets obtained with HTS.

### REFERENCES

K. Sridharan, N. J. Gogtay, Therapeutic nucleic acids: current clinical status, Br J Clin Pharmacol 82(3) (2016) 659-72.
A. Goyon, P. Yehl, K. Zhang, Characterization of therapeutic oligonucleotides by liquid chromatography, J Pharm Biomed Anal 182 (2020) 113105.
C. Chakraborty, A. R. Sharma, G. Sharma, C. G. P. Doss, S. S. Lee, Therapeutic miRNA and siRNA: Moving from Bench to Clinic as Next Generation Medicine, Mol Ther Nucleic Acids 8 (2017) 132-143.
C. I. E. Smith, R. Zain, Therapeutic Oligonucleotides: State of the Art, Annu Rev Pharmacol Toxicol 59 (2019) 605-630.
D. Adams, A. Gonzalez-Duarte, W. D. O'Riordan, C. C. Yang, M. Ueda, A. V. Kristen, I. Tournev, H. H. Schmidt, T. Coelho, J. L. Berk, K. P. Lin, G. Vita, S. Attarian, V. Plante-Bordeneuve, M. M. Mezei, J. M. Campistol, J. Buades, T. H. Brannagan, 3rd, B. J. Kim, J. Oh, Y. Parman, Y. Sekijima, P. N. Hawkins, S. D. Solomon, M. Polydefkis, P. J. Dyck, P. J. Gandhi, S. Goyal, J. Chen, A. L. Strahs, S. V. Nochur, M. T. Sweetser, P. P. Garg, A. K. Vaishnaw, J. A. Gollob, O. B. Suhr, Patisiran, an RNAi Therapeutic, for Hereditary Transthyretin Amyloidosis, N Engl J Med 379(1) (2018) 11-21.
U. Sahin, K. Kariko, O. Tureci, mRNA-based therapeutics—developing a new class of drugs, Nat Rev Drug Discov 13(10) (2014) 759-80.
U. Sahin, E. Derhovanessian, M. Miller, B. P. Kloke, P. Simon, M. Lower, V. Bukur, A. D. Tadmor, U. Luxemburger, B. Schrors, T. Omokoko, M. Vormehr, C. Albrecht, A. Paruzynski, A. N. Kuhn, J. Buck, S. Heesch, K. H. Schreeb, F. Muller, I. Ortseifer, I. Vogler, E. Godehardt, S. Attig, R. Rae, A. Breitkreuz, C. Tolliver, M. Suchan, G. Martic, A. Hohberger, P. Sorn, J. Diekmann, J. Ciesla, O. Waksmann, A. K. Bruck, M. Witt, M. Zillgen, A. Rothermel, B. Kasemann, D. Langer, S. Bolte, M. Diken, S. Kreiter, R. Nemecek, C. Gebhardt, S. Grabbe, C. Holler, J. Utikal, C. Huber, C. Loquai, O. Tureci, Personalized RNA mutanome vaccines mobilize poly-specific therapeutic immunity against cancer, Nature 547(7662) (2017) 222-226.
N. Pardi, M. J. Hogan, F. W. Porter, D. Weissman, mRNA vaccines—a new era in vaccinology, Nat Rev Drug Discov 17(4) (2018) 261-279.
K. A. Dowd, S. Y. Ko, K. M. Morabito, E. S. Yang, R. S. Pelc, C. R. DeMaso, L. R. Castilho, P. Abbink, M. Boyd, R. Nityanandam, D. N. Gordon, J. R. Gallagher, X. Chen, J. P. Todd, Y. Tsybovsky, A. Harris, Y. S. Huang, S. Higgs, D. L. Vanlandingham, H. Andersen, M. G. Lewis, R. De La Barrera, K. H. Eckels, R. G. Jarman, M. C. Nason, D. H. Barouch, M. Roederer, W. P. Kong, J. R. Mascola, T. C. Pierson, B. S. Graham, Rapid development of a DNA vaccine for Zika virus, Science 354(6309) (2016) 237-240.
J. M. Richner, S. Himansu, K. A. Dowd, S. L. Butler, V. Salazar, J. M. Fox, J. G. Julander, W. W. Tang, S. Shresta, T. C. Pierson, G. Ciaramella, M. S. Diamond, Modified mRNA Vaccines Protect against Zika Virus Infection, Cell 168(6) (2017) 1114-1125 e10.
A. Patel, E. L. Reuschel, K. A. Kraynyak, T. Racine, D. H. Park, V. L. Scott, J. Audet, D. Amante, M. C. Wise, A. A. Keaton, G. Wong, D. O. Villarreal, J. Walters, K. Muthumani, D. J. Shedlock, M. A. de La Vega, R. Plyler, J. Boyer, K. E. Broderick, J. Yan, A. S. Khan, S. Jones, A. Bello, G. Soule, K. N. Tran, S. He, K. Tierney, X. Qiu, G. P. Kobinger, N. Y. Sardesai, D. B. Weiner, Protective Efficacy and Long-Term Immunogenicity in Cynomolgus Macaques by Ebola Virus Glycoprotein Synthetic DNA Vaccines, J Infect Dis 219(4) (2019) 544-555.
M. D. Shin, S. Shukla, Y. H. Chung, V. Beiss, S. K. Chan, O. A. Ortega-Rivera, D. M. Wirth, A. Chen, M. Sack, J. K. Pokorski, N. F. Steinmetz, COVID-19 vaccine development and a potential nanomaterial path forward, Nat Nanotechnol (2020).
J. Wang, Z. Lu, M. G. Wientjes, J. L. Au, Delivery of siRNA therapeutics: barriers and carriers, The AAPS journal 12(4) (2010) 492-503.
M. Durymanov, J. Reineke, Non-viral Delivery of Nucleic Acids: Insight Into Mechanisms of Overcoming Intracellular Barriers, Front Pharmacol 9 (2018) 971.
A. Akinc, M. A. Maier, M. Manoharan, K. Fitzgerald, M. Jayaraman, S. Barros, S. Ansell, X. Du, M. J. Hope, T. D. Madden, B. L. Mui, S. C. Semple, Y. K. Tam, M. Ciufolini, D. Witzigmann, J. A. Kulkarni, R. van der Meel, P. R. Cullis, The Onpattro story and the clinical translation of nanomedicines containing nucleic acid-based drugs, Nat Nanotechnol 14(12) (2019) 1084-1087.
A. C. Anselmo, S. Mitragotri, Nanoparticles in the clinic: An update, Bioeng Transl Med 4(3) (2019) e10143.
Liposome drug products: chemistry, manufacturing, and controls; human pharmacokinetics and bioavailability; and labeling documentation, U. S. Food and Drug Administration, 2018.
T. M. Allen, C. Hansen, F. Martin, C. Redemann, A. Yau-Young, Liposomes containing synthetic lipid derivatives of poly(ethylene glycol) show prolonged circulation half-lives in vivo, Biochim Biophys Acta 1066(1) (1991) 29-36.
O. Garbuzenko, Y. Barenholz, A. Priev, Effect of grafted PEG on liposome size and on compressibility and packing of lipid bilayer, Chem Phys Lipids 135(2) (2005) 117-29.
M. L. Immordino, F. Dosio, L. Cattel, Stealth liposomes: review of the basic science, rationale, and clinical applications, existing and potential, Int J Nanomedicine 1(3) (2006) 297-315.
A. Schroeder, C. G. Levins, C. Cortez, R. Langer, D. G. Anderson, Lipid-based nanotherapeutics for siRNA delivery, J Intern Med 267(1) (2010) 9-21.
P. R. Cullis, M. J. Hope, Lipid Nanoparticle Systems for Enabling Gene Therapies, Mol Ther 25(7) (2017) 1467-1475.
M. Johnsson, K. Edwards, Liposomes, disks, and spherical micelles: aggregate structure in mixtures of gel phase phosphatidylcholines and poly(ethylene glycol)-phospholipids, Biophys J 85(6) (2003) 3839-47.
K. K. Gill, A. Kaddoumi, S. Nazzal, PEG-lipid micelles as drug carriers: physiochemical attributes, formulation principles and biological implication, J Drug Target 23(3) (2015) 222-31.
K. Yang, J. T. Delaney, U. S. Schubert, A. Fahr, Fast high-throughput screening of temoporfin-loaded liposomal formulations prepared by ethanol injection method, J Liposome Res 22(1) (2012) 31-41.
M. A. Schubert, C. C. Muller-Goymann, Solvent injection as a new approach for manufacturing lipid nanoparticles—evaluation of the method and process parameters, Eur J Pharm Biopharm 55(1) (2003) 125-31.
A. A. Mokhtarieh, J. Lee, S. Kim, M. K. Lee, Preparation of siRNA encapsulated nanoliposomes suitable for siRNA delivery by simply discontinuous mixing, Biochim Biophys Acta Biomembr 1860(6) (2018) 1318-1325.
C. Jaafar-Maalej, R. Diab, V. Andrieu, A. Elaissari, H. Fessi, Ethanol injection method for hydrophilic and lipophilic drug-loaded liposome preparation, J Liposome Res 20(3) (2010) 228-43.
I. V. Zhigaltsev, N. Belliveau, I. Hafez, A. K. Leung, J. Huft, C. Hansen, P. R. Cullis, Bottom-up design and synthesis of limit size lipid nanoparticle systems with aqueous and triglyceride cores using millisecond microfluidic mixing, Langmuir 28(7) (2012) 3633-40.
S. Sabnis, E. S. Kumarasinghe, T. Salerno, C. Mihai, T. Ketova, J. J. Senn, A. Lynn, A. Bulychev, I. McFadyen, J. Chan, O. Almarsson, M. G. Stanton, K. E. Benenato, A Novel Amino Lipid Series for mRNA Delivery: Improved Endosomal Escape and Sustained Pharmacology and Safety in Non-human Primates, Mol Ther 26(6) (2018) 1509-1519.
S. C. Semple, A. Akinc, J. Chen, A. P. Sandhu, B. L. Mui, C. K. Cho, D. W. Sah, D. Stebbing, E. J. Crosley, E. Yaworski, I. M. Hafez, J. R. Dorkin, J. Qin, K. Lam, K. G. Rajeev, K. F. Wong, L. B. Jeffs, L. Nechev, M. L. Eisenhardt, M. Jayaraman, M. Kazem, M. A. Maier, M. Srinivasulu, M. J. Weinstein, Q. Chen, R. Alvarez, S. A. Barros, S. De, S. K. Klimuk, T. Borland, V. Kosovrasti, W. L. Cantley, Y. K. Tam, M. Manoharan, M. A. Ciufolini, M. A. Tracy, A. de Fougerolles, I. MacLachlan, P. R. Cullis, T. D. Madden, M. J. Hope, Rational design of cationic lipids for siRNA delivery, Nature biotechnology 28(2) (2010) 172-6.
N. M. Belliveau, J. Huft, P. J. Lin, S. Chen, A. K. Leung, T. J. Leaver, A. W. Wild, J. B. Lee, R. J. Taylor, Y. K. Tam, C. L. Hansen, P. R. Cullis, Microfluidic Synthesis of Highly Potent Limit-size Lipid Nanoparticles for In Vivo Delivery of siRNA, Mol Ther Nucleic Acids 1 (2012) e37.
D. van Swaay, A. deMello, Microfluidic methods for forming liposomes, Lab Chip 13(5) (2013) 752-67.
E. Yamamoto, K. Hyodo, N. Ohnishi, T. Suzuki, H. Ishihara, H. Kikuchi, N. Asakawa, Direct, simultaneous measurement of liposome-encapsulated and released drugs in plasma by on-line SPE-SPE-HPLC, J Chromatogr B Analyt Technol Biomed Life Sci 879(30) (2011) 3620-5.
L. Li, J. P. Foley, R. Helmy, Simultaneous separation of small interfering RNA and lipids using ion-pair reversed-phase liquid chromatography, J Chromatogr A 1601 (2019) 145-154.

1.-35. (canceled)

36. A high-throughput method for optimizing the process for manufacturing a lipid nanoparticle (LNP) preparation comprising:
a. obtaining a first solution comprising an aqueous phase;
b. obtaining a second solution comprising an organic phase and a plurality of molecules capable of self-assembly, and wherein said first and second solutions are intermixable;
c. dissolving at least one payload molecule into either the first or second solution;
d. using a robotic liquid handler to prepare and dispense said phases with varied compositions into a plurality of wells;
e. mixing said first and second solutions to obtain lipid nanoparticles encapsulating said payload using said robotic liquid handler under conditions suitable for LNP formation; wherein at least one of the following conditions are varied amongst different wells: type of self-assembly molecule, composition ratio of said self-assembly molecule; ratio and/or concentration of said self-assembly molecule to said payload, the selection of phase, buffer type and pH, the injection sequence, injection speed, mixing speed, volume, phase ratio, injection duration, and mixing duration;
f. measuring at least one of the following: encapsulation efficiency, particle size distribution, purification and particle recovery rate, and formulation stability of said LNPs;
g. determining the optimal parameters for manufacturing said LNP preparation; and
h. manufacturing said LNP preparation based on said optimal parameters.

37. The method of claim 36, wherein the payload is an oligonucleotide.

38. The method of claim 37, wherein the oligonucleotide is an antisense molecule, a siRNA, a shRNA, or a mRNA.

39.-43. (canceled)

44. The method of claim 36, wherein the payload is a polypeptide or a small molecule.

45. The method of claim 44, wherein said polypeptide is between about 1,000 Da and about 10,000 Da.

46. (canceled)

47. (canceled)

48. The method of claim 36, wherein the payload is dissolved in the first solution or in the second solution.

49. (canceled)

50. The method of claim 36, wherein the first solution is an aqueous buffer, or wherein the first solution comprises pH- and osmolality-controlled buffers.

51. (canceled)

52. The method of claim 36, wherein the organic phase of the second solution comprises methanol or ethanol.

53. (canceled)

54. The method of claim 36, wherein the self-assembly molecules include at least a lipid component comprised of at least one species of lipid molecule, wherein the at least one species of lipid molecule is selected from a cationic lipid species, a non-cationic lipid species, and a phospholipid species.

55. (canceled)

56. The method of claim 54, wherein said second solution comprises more than one type of lipid.

57. The method of claim 54, wherein:
a. the total concentration of lipid is varied; or
b. wherein the percentage of lipids that are PEGylated is varied.

58. The method of claim 57, wherein the total concentration of lipid is varied between about 0.4 and about 4 mM, or wherein the percentage of lipids that are PEGylated are varied between about 0.5% to about 5% of the total lipid composition.

59. (canceled)

60. (canceled)

61. The method of claim 37, wherein the payload's N:P ratio is varied between about 0.5 to about 5.

62. (canceled)

63. The method of claim 36, wherein the LNP is:
a. a polymer lipid nanoparticle,
b. a liposome; or
c. a lipoprotein nanoparticle.

64. (canceled)

65. (canceled)

66. The method of claim 36, wherein:
a. said first solution is injected into said second solution, or
b. said second solution is injected into said first solution.

67. (canceled)

68. The method of claim 36, wherein:
a. the optimal parameters are those which produce an encapsulation efficiency of the payload greater than 80%;
b. the optimal parameters are those which produce a LNP with a mean diameter of 80-200 nm, having a unimodal size distribution, and a polydispersity of less than about 30%; or
c. the LNPs maintain a similar size distribution and payload encapsulation for at least one month under storage in solution at 4 degrees Celsius.

69.-105. (canceled)

106. A method of administering a LNP preparation to a patient in need thereof, wherein said LNP preparation is manufactured by:
a. obtaining a first solution comprising an aqueous phase;
b. obtaining a second solution comprising an organic phase and a plurality of molecules capable of self-assembly, and wherein said first and second solutions are intermixable;
c. dissolving at least one payload molecule into either the first or second solution;
d. using a robotic liquid handler to prepare and dispense said phases with varied compositions into a plurality of wells;
e. mixing said first and second solutions to obtain lipid nanoparticles encapsulating said payload using said robotic liquid handler under conditions suitable for LNP formation; wherein at least one of the following conditions are varied amongst different wells: type of self-assembly molecule, composition ratio of said self-assembly molecule; ratio and/or concentration of said self-assembly molecule to said payload, the selection of phase, buffer type and pH, the injection sequence, injection speed, mixing speed, volume, phase ratio, injection duration, and mixing duration;
f. measuring at least one of the following: encapsulation efficiency, particle size distribution, purification and particle recovery rate, and formulation stability of said LNPs;
g. determining the optimal parameters for manufacturing said LNP preparation; and
h. manufacturing said LNP preparation based on said optimal parameters.

107. The method of claim 106, wherein the payload is an oligonucleotide, a polypeptide, or a small molecule.

108.-175. (canceled)

176. An optimized lipid nanoparticle (LNP) manufactured by a process comprising the following steps:
a. obtaining a first solution comprising an aqueous phase;
b. obtaining a second solution comprising an organic phase and a plurality of molecules capable of self-assembly, and wherein said first and second solutions are intermixable;
c. dissolving at least one payload molecule into either the first or second solution;
d. using a robotic liquid handler to prepare and dispense said phases with varied compositions into a plurality of wells;
e. mixing said first and second solutions to obtain lipid nanoparticles encapsulating said payload using said robotic liquid handler under conditions suitable for LNP formation; wherein at least one of the following conditions are varied amongst different wells: type of self-assembly molecule, composition ratio of said self-assembly molecule; ratio and/or concentration of said self-assembly molecule to said payload, the selection of phase, buffer type and pH, the injection sequence, injection speed, mixing speed, volume, phase ratio, injection duration, and mixing duration;
f. measuring at least one of the following: encapsulation efficiency, particle size distribution, purification and particle recovery rate, and formulation stability of said LNPs;
g. determining the optimal parameters for manufacturing said LNP preparation; and
h. manufacturing said LNP preparation based on said optimal parameters.

177. The optimized LNP of claim 176, wherein the payload is an oligonucleotide, a polypeptide, or a small molecule.

178.-209. (canceled)

210. The optimized LNP of claim 176, wherein the LNPs maintain a similar size distribution and payload encapsulation for at least one month under storage in solution at 4 degrees Celsius.

211. A workflow for HTS screening of a plurality of parameters for LNP formation, comprising:
(i) a robotic liquid handler;
(ii) at least one instrument capable of measuring desired LNP characteristics; and
(iii) at least one microplate comprising a plurality of microwells;
wherein said robotic liquid handler is capable of injecting a plurality of solutions into each of said microwells;
wherein said parameters are systematically varied between microwells; and
wherein said desired LNP characteristics are capable of being measured for each microwell.

212.-214. (canceled)

215. The method of claim 36,
wherein the encapsulation efficiency is optimized by measuring the charge ratio of ionizable lipid/oligonucleotide.

216.-238. (canceled)
